(19) United States
(12) Patent Application Publication
(10) Pub. No.: US 2023/0157533 A1
(43) Pub. Date: May 25, 2023

(54) A COMPUTER-IMPLEMENTED SYSTEM AND METHOD FOR ASSESSING A LEVEL OF ACTIVITY OF A DISEASE OR CONDITION IN A PATIENT'S EYE

(71) Applicant: Novartis AG, Basel (CH)

(72) Inventors: Hsun-Hsien Shane CHANG, Brookline, MA (US); Daniel François Claude Lorand, Dietwiller (FR); Zufar MULYUKOV, Reinach (CH)

(21) Appl. No.: 17/916,980

(22) PCT Filed: Apr. 26, 2021
(86) PCT No.: PCT/IB2021/053427

§ 371 Date: Oct. 4, 2022

Related U.S. Application Data
(60) Provisional application No. 63/017,341, filed on Apr. 29, 2020.

(51) Int. Cl. A61B 3/00; G16H 50/30; G16H 20/10; G16H 50/50; A61B 3/12; A61B 3/10

(52) U.S. Cl. CPC A61B 3/0025; G16H 50/30; G16H 20/10; G16H 50/50; A61B 3/1225; A61B 3/102

(57) ABSTRACT

The present invention relates to computer-implemented method for assessing a level of activity, including presence or an absence, of a disease in at least one eye of a patient, wherein the disease is a neovascular ocular disease. The method comprises the steps of receiving, via one or more input elements, a set of input patient data corresponding to the patient and comprising retinal images of the patient; applying a first algorithm for imaging data analysis to the retinal images to identify values of e anatomical variables of the patient's eye; applying a second algorithm to the values of anatomical variables identified, and to distinct clinical, non-image derived input patient data comprised in the set of input patient data, in order to consequently make an assessment of the level of activity of the disease in the eye of the patient, and/or of the progression or regression of the disease with respect to a level of activity formerly determined. Based on the assessment, a disease activity score is generated and output corresponding to the level of activity of the disease. The assessment of disease activity is used to adjust a dosing regimen of a drug for treatment of the patient's eye disease.

### TECHNICAL FIELD

The present invention relates a computer-implemented method for automatically assessing a level of activity of a disease or of a condition in a patient's eye, for example wherein the disease is a neovascular ocular disease causing neovascularization of a retina and/or in proximity of a retina of the eye.

Based on the assessment, the method can consequently automatically output information on level of disease activity, features related to such disease activity, and/or optimal timing of medical interventions on the eye, such as of drug injections for treatment of the disease or condition. The information can comprise dosing frequency, timing of patient visit for carrying our next intervention etc., as per approved drug posology.

In particular, the computer-implemented method according to the present invention generates and outputs, based on the assessment, a disease activity score corresponding to the level of activity of the disease, wherein the disease activity score can be also linked to a probability, or to the appropriateness, of effectively switching from a current dosing regimen for the drug used for treatment of the patient's eye disease, to a different dosing regimen thereof.

The present invention also relates to a computing system, designed to carry out the method, wherein the computing system comprises a computing device including one or more processors; one or more input elements; memory; and one or more programs stored in the memory including instructions for implementing the method.

The present invention further relates to a non-transitory computer-readable storage medium storing one or more programs configured to be executed by one or more processors of an electronic device with one or more input elements, the one or more programs including instructions for carrying out the above mentioned computer implemented method.

The computer-implemented method according to the present invention is suitable to train machine learning algorithms to assess disease activity in patients affected by neovascularization of an eye's retina and/or in proximity of the retina, particularly by age-related macular degeneration, and especially by wet age-related macular degeneration, also designatable as w-AMD. A definition of neovascular ocular disease in the sense of the present invention is given below in the Background Art section.

Based on the assessment, the algorithms underlying the computer-implemented method according to the present invention support a clinical decision-enabling dosing system which provides a precise and intelligent disease activity assessment and drug dosing frequency guidance to health care providers or physicians treating such retina-affecting diseases, especially for treatment of wet age-related macular degeneration (w-AMD) by anti-vascular endothelial growth factor (anti-VEGF) drugs.

### Background Art

Retinopathies in general encompass several retinal vascular diseases which may ultimately lead to vision impairment. In particular, treatment of neovascular ocular diseases causing neovascularization of a retina and/or in proximity of a retina of the eye is addressed in the following. Among these, neovascular age-related macular degeneration, or nAMD, also known as “exudative” or “wet” AMD, otherwise indicated by w-AMD, is a chronic eye disorder characterized by the abnormal formation of new choroidal vessels with growth under the retinal pigment epithelium (RPE) or in subretinal spaces, impacting the central area of the retina in the eye, called the macula which is responsible for central vision and seeing detail. This results in blurred vision, wavy lines, dull colors, blind spots and ultimately in severe vision loss.

Other retinopathies comprise, by way of a non exhaustive example, diabetic retinopathy (DR); diabetic macular edema (DME); myopic choroidal neovascularization (mCNV); macular edema following retinal vein occlusion (RVO); retinopathy of prematurity (ROP).

The term “neovascular ocular disease”, as used herein, refers to a condition, disease, or disorder associated with ocular neovascularization. A “neovascular ocular disease” that can be treated using a method of the disclosure includes, a condition, disease, or disorder associated with ocular neovascularization, including, but not limited to, abnormal angiogenesis, choroidal neovascularization (CNV), choroidal neovascularization (CNV) associated with w-AMD, retinal vascular permeability, retinal edema, diabetic retinopathy (particularly proliferative diabetic retinopathy (PDR) and non-proliferative diabetic retinopathy (NPDR)), macular edema (ME), diabetic macular edema (DME), neovascular (exudative) age-related macular degeneration (w-AMD), sequela associated with retinal ischemia, Retinal Vein Occlusion (RVO), Central Retinal Vein Occlusion (CRVO), Branch Retinal Vein Occlusion (BRVO), macular edema following retinal vein occlusion, and posterior segment neovascularization.

Patients affected by the above retinopathies are nowadays treated especially by injections in the affected eye of anti-vascular endothelial cell growth factor, or anti-VEGF, drugs, also called VEGF inhibitors. Anti-vascular endothelial cell growth factor drugs comprise, for instance ranibizumab, aflibercept and brolucizumab-dbII, the latter being particularly suited for treatment of wet age-related macular degeneration, or w-AMD.

Patients on anti-VEGF therapy for treating the above retinopathies require regular visits to healthcare professionals for disease monitoring and re-treatment. Further, the pattern of disease activity, such as w-AMD disease activity, and of correlated required administrations of anti-VEGF drug by injection, is hard to predict at patient level.

The fact that some acting anti-VEGF agents are long-acting makes monitoring of patients treated thereby difficult. Also, it is difficult to predict how effective a switch from a current drug dosing regimen to a different dosing regimen will be.

In many cases, these issues result in the inadequate treatment of retinopathies such as w-AMD in patients and/or in an increased burden on patients, e.g. in the form of unnecessary injections and/or of superfluous healthcare professional visits.

To date, there are many challenges to managing w-AMD patients, or patients affected by one of the above-mentioned retinopathies, in real-world practice. Ophthalmologists have limited time to provide consultation to these patients during regular visits and often need to make quick, not fully well-informed dosing decisions. Information to aid clinicians in making dosing decisions is typically not readily available e.g. because of limited image segmentation and volumetry of retinal fluids and/or no quantification of disease activity. At any rate, disease activity assessment and dosing decisions can be complex and there may be variations among different ophthalmologists regarding what constitutes optimal dosing in individual cases, as well as patient to patient phenotypic variations.

There is currently no established, rationalized computer-based, fully automated procedure for managing treatment of patients affected by w-AMD or other retinopathies, improving their treatment while also alleviating the related burden of repeated visits which impact patients' independence and quality of life.

Therefore, there is a need for a method that reliably and accurately assesses disease activity of w-AMD and/or of other retinopathies and that provides patient-specific anti-VEGF treatment regimen models, such as customized dosing frequency models.

### DISCLOSURE OF THE INVENTION

According to the invention, this need is settled by a computer-implemented method for assessing a level of activity, including presence or an absence, of a disease or of a condition in at least one eye of a patient, wherein the disease is a neovascular ocular disease.

Such assessment of a disease activity level is automatically carried out by a computer-implemented system and by the use of a non-transitory computer-readable storage medium. Each of the foregoing is defined by one or more appended Claims.

The method according to the present invention allows to optimally individualize treatment intervals by administration of a prescribed drug, e.g. by injection in the affected eye of the patient, in order to better preserve patient's vision and to concurrently promote the patient's independence and better quality of life.

By optimizing decisions at point of care through an easy-to-use expert system emulating the decision making of highly experienced retinal specialists, the patient's vision can be preserved efficiently with lowest patient and health care professional burden. All relevant information to guide in disease activity assessments and dosing decisions can also be advantageously centralized.

As mentioned, the method can be implemented on one or more computing devices, including one or more processors, a memory and one or more input and/or output elements.

The main output of the method, and of the models generated by its implementation, is a disease activity score corresponding to the level of activity of the disease to be treated. Such disease activity score, or DA score, is used to evaluate the level of disease activity which can be further categorized into high or medium or low.

The disease activity score can be considered as an index that measures the extent to which potentially reversible aspects of a disease to be treated are present in the patient, as detectable based on input patient data such as values of one or more anatomical and functional variables of the patient.

The disease activity score can be further linked to the appropriateness of switching from a current dosing regimen of the drug for treatment of the patient's eye disease to a different dosing regimen thereof. Thus, the disease activity score can also be associated to the expected success or failure of such a dosing regimen switch, in terms of achieving respectively a lower or higher disease activity score by the dosing regimen switch.

The method according to the present invention can therefore yield not only a score correlated to the current disease activity, but also a prediction of disease activity change as a result of a change from a current dosing regimen of the drug for treatment of the patient's eye disease to a different dosing regimen thereof, that is a prediction of disease activity under a more frequent or less frequent dosing regimen with respect to the current one.

The disease activity score can be output by embodiments of methods of the present invention as a numerical value that characterizes the level of disease activity, e.g. to be interpreted by a health professional provided with a reference list of disease activity scores; or as a warning message aimed at a health care professional or alternatively stating a need to review the current drug dosing regimen; or as an alert triggered and issued only once the calculated level exceeds a pre-set threshold. The disease activity score, and possibly a confidence or uncertainty statistical value associated thereto, can be visualized, for instance, on a display screen of a device connected to the computer system implementing the present method, preferably by aid of graphical user interfaces that intuitively convey the disease activity level and guide dosing decisions taken by health care professionals.

The method according to the present invention comprises the step of receiving, via the one or more input elements, a set of input patient data corresponding to the patient. The set of input patient data comprises at least one or more retinal images of the patient, preferably optical coherence tomography (OCT) images of the patient's eyes.

The above-mentioned set of input patient data comprises real-world data of a specific patient for whom the level of disease activity in connection with one of the above given retinopathies needs to be assessed. Beyond retinal images, in embodiments of the invention, set of input patient data can further comprise clinical, non-imaging derived input patient data such as: longitudinal patient eye data e.g. best corrected visual acuity; and/or patient medical history information and/or patient longitudinal data capturing some physiological characteristics of the specific patient not necessarily directly correlated to eye condition; and/or baseline demographic data such as age, weight, gender, race etc. of the examined patient. Longitudinal data referring to a patient can be data of the monitored patient collected over a period of time, e.g. from week 0 to a target week 16 or 20 following treatment initiation, for instance in 2 or 4 week intervals.

In general, the set of input patient data will mirror a multiplicity of input variables, or features or parameters, suitable to describe a patient's health condition, which variables or features or parameters are embodied or stored in algorithms implemented by the method according to the present invention to assess the level of activity, including presence or an absence, of a disease in at least one eye of the patient's eyes.

In embodiments of the invention, a set of input patient data may include at least a diagnosis of a disease or condition affecting the retina of the patient's eye at a primary or secondary care service, made at a first index date prior to the assessment date. Such disease or condition can be one or more of the diseases or conditions listed in the following: wet age-related macular degeneration, also designatable as w-AMD; diabetic retinopathy, also designatable as DR; diabetic macular edema, also designatable as DME; myopic choroidal neovascularization, also designatable as mCNV; macular edema following retinal vein occlusion, also designatable as RVO; retinopathy of prematurity, also designatable as ROP.

Examples of longitudinal patient data input types include: best corrected visual acuity (BCVA); central subfield foveal thickness (CSFT); drusen area and/or volume; drusen probability; epiretinal membrane thickness; epiretinal membrane probability; fibrous pigment epithelium detachment probability; geographic atrophy area and/or volume; geographic atrophy probability; healthy probability (no abnormality or biomarker detected); hyperreflective focii and hard exudates probability; outer retinal atrophy area and/or volume; outer retinal atrophy probability; reticular pseudo-drusen area and/or volume; reticular pseudo-drusen probability; ganglion cell layer and inner plexiform layer volume, in a predetermined area (e.g., a 1 mm, 3 mm, and/or 6 mm area); inner nuclear layer and outer plexiform layer volume, in a predetermined area (e.g., a 1 mm, 3 mm, and/or 6 mm area); intraretinal fluid and cysts volume, in a predetermined area (e.g., a 1 mm, 3 mm, and/or 6 mm area); outer nuclear layer volume, in a predetermined area (e.g., a 1 mm, 3 mm, and/or 6 mm area); pigment epithelium detachment volume, in a predetermined area (e.g., a 1 mm, 3 mm, and/or 6 mm area); photoreceptors and retinal pigment epithelium volume, in a predetermined area (e.g., a 1 mm, 3 mm, and/or 6 mm area); retinal nerve fiber layer volume, in a predetermined area (e.g., a 1 mm, 3 mm, and/or 6 mm area); and subretinal fluid volume, in a predetermined area (e.g., a 1 mm, 3 mm, and/or 6 mm area).

As mentioned, in embodiments of the invention the input patient data can be structured according to a chronological sequence, e.g. in preset time periods prior to a target assessment date. For instance, during the monitoring of a treated patient, time periods of 0, 4, 8, 12 weeks, up to a target assessment date fixed, by way of example, at 16 weeks from beginning of the patient's treatment, can be considered. Such preset time periods can capture and account for possible patterns and dependencies during treatment.

In some embodiments, values for one or more of the above patient data inputs are determined or derived from one or more other data input values. In some examples, one or more values for one or more of the above data inputs are based on received OCT images, e.g., based on dimensions of anatomical features captured in the OCT images.

The OCT images can also be generated by an OCT device situated in a clinician's office or in the patient's home. In various embodiments, as an enhancement to standard OCT, the images can be generated, for example, by a spectral domain optical coherence tomography (SD-OCT) imaging device.

The method according to the present invention further comprises the step of applying a first algorithm for imaging data analysis to the one or more retinal images received as part of the set of input patient data.

In some embodiments, the first algorithm can be a machine learning model or artifact for image segmentation, for instance generated by applying a machine learning algorithm to a historical set of patient image data. However, the first algorithm can also be alternatively be a conventional, explicitly coded algorithm developed without machine learning aid.

The method according to the present invention further comprises the step of identifying, based on patient's retinal images received, values of one or more anatomical variables, or features, of the patient's eye.

By way of example, the values of such anatomical variables can be any combination of: values of central retinal thickness and/or volume; of inter-retinal fluid volume; of sub-retinal fluid volume; of pigment epithelial detachment, also indicated as PED; of drusenoid, fibrovascular, or serous PED; of hyperreflective foci; of ellipsoid zone defect; of external limiting membrane band defect; of retinal pigment epithelial atrophy.

Other anatomical variables whose values can be identified to be taken into account in the implementation of the present method can be: central subfield foveal thickness (CSFT); ganglion cell layer and inner plexiform layer; inner nuclear layer and outer plexiform layer volume; cysts volume; outer nuclear layer volume; pigment epithelium detachment volume; photoreceptors and retinal pigment volume; retinal nerve fiber layer volume.

In some examples, fluid is a useful biomarker of disease activity in w-AMD, therefore, in principle, in some examples fluid may serve as a basis for diagnosis and management recommendations. Examples include intraretinal fluid (IRF), subretinal fluid (SRF) and/or subretinal pigment epithelium (RPE) fluid. In embodiments of the invention, fluid may be a variable or feature to be taken into account when assessing the level of disease activity and making a decision to maintain a fixed treatment regimen or move to a treat and extend dosing schedule.

In some embodiments, retinal fluid, disease activity assessment and treatment frequency exhibit a correlation.

In some embodiments, retinal thickness—especially thick or abnormally thin retinas—may be a factor in disease activity assessment and treatment considerations and outcome determination. Retinal thickness can be a very important variable or feature with considerable impact on ultimate algorithm output.

The method according to the present invention further comprises the step of applying a second algorithm to the values of the one or more anatomical variables identified, and to distinct clinical, non-image derived input patient data comprised in the set of input patient data. Examples of distinct clinical, non-image derived input patient data have been provided above.

The application of the second algorithm allows to take into account levels and changes, for a given patient, of the identified anatomical variables and/or of distinct non-image derived patient data, with respect to visits of the same patient previous to the current assessment.

The application of the second algorithm can therefore achieve to estimate visual acuity loss or gain; rate of retinal thickness loss or gain; rate of intraretinal fluid volume loss or gain.

The second algorithm can also be a machine learning model or artifact, generated by training one or more machine learning algorithms on historical sets of patient data which include at least values for the one or more identified anatomical variables, for instance derived from clinical trials. As mentioned, the values for the one or more identified anatomical variables are preferably complemented with values of historic patient data such as patient demographics and/or medical history and/or concomitant medications, and/or comorbidities and/or adverse events and/or serious adverse events.

Accordingly, in some embodiments, the second algorithm is a disease activity assessment model generated by one or more machine learning algorithms comprising a multiplicity of input variables corresponding to the one or more identified anatomical variables and to the distinct clinical, non-image derived input patient data. The one or more machine learning algorithms are trained on a historical set of patient data from a plurality of historical patients diagnosed with the disease which is being assessed.

In such cases, the historical set of patient data include input values for the one or more identified anatomical variables, recognized and quantified out of retinal images of each of a multiplicity of historical patients enrolled in dedicated clinical trials conducted, for instance, to ascertain the working of anti-VEGF drugs; and/or include values correlated to the above-mentioned clinical, non-image derived input patient data of each of the multiplicity of historical patients, comprising historical patients' demographics and/or medical history and/or concomitant medication and/or comorbidities and/or adverse events and/or serious adverse events. The clinical trials can be run traditionally and/or remotely via devices and/or sensors collecting patient data.

In some embodiments, the historical set of patient data comprises input values extracted from at least one of clinical trial data and anonymized real-world patient data from commercially-available databases; or alternatively from electronic medical records kept by heath care providers and/or from other electronic health registers kept by at least a health authority and/or by a similar institution; and/or from sociodemographic databases.

In some embodiments, the second algorithm can be updated based on a further historical set of patient data from a further plurality of historical patients diagnosed with the disease to be assessed. In this case, the further historical set of patient data includes values for the one or more identified anatomical variables, derived from a further set of one or more retinal images, e.g. OCT retinal images, of each of the further plurality of historical patients. The further historical set of patient data preferably includes also values correlated to the further plurality of historical patients' demographics and/or medical history and/or concomitant medication and/or comorbidities and/or adverse events and/or serious adverse events, as derivable e.g. from Real World and/or Randomized Clinical Trials patient data.

In some embodiments, such a step of updating the second algorithm comprises the step of re-training the one or more machine learning algorithms by complementing the historical set of patient data with the further historical set of patient data. Accordingly, an updated disease activity assessment model is generated with the one or more re-trained machine learning algorithms.

In some embodiments, the generated disease assessment model is updated in real time. In this case, the further historical set of patient data preferably comprises real-world patient data as obtained from the assessment of the level of activity of the disease of interest and/or of the progression or regression of the disease in corresponding real-world patients. Therefore, the further historical set of patient data can originate from the visits carried out by health care providers, when the disease activity assessment model is run on a set of input patient data corresponding to real-world visited patients. In these instances, the real-world patient data can comprise updated anatomical variable data, such as change in anatomical variable measurements, over a period of time.

Based on the application of the second algorithm to values of imaging-identified anatomical variables and to different clinical, non-image derived input patient data as above elucidated—for instance following a preliminary step of training the second algorithm to generate a corresponding disease activity assessment model—an assessment is made of the level of activity of the disease in the at least one eye of the patient.

In some embodiments, based on the application of the second algorithm, in addition to or alternatively to assessing the disease activity level, the method according to the present invention can assess the progression or regression of the disease with respect to a level of activity formerly determined.

The assessment of disease activity corresponds to a dosing regimen of a drug for treatment of the patient's eye disease. The obtained disease activity assessment can thus be employed to adjust or modify the dosing regimen.

Based on the assessment, the method according to the present invention generates and outputs, via the one or more output elements, a disease activity score corresponding to the level of activity of the disease, as above introduced.

In some embodiments, the generated model is qualified and/or validated. After qualification and/or validation, the disease activity assessment model is provided to health care professionals, e.g. as a front end cloud-based computer program. Thus, the health care professionals may input a specific patient's data into the model, to receive a disease activity score corresponding to the level of activity of the disease in the patient examined. The disease activity score allows to adopt accurate and medically relevant treatment options to mitigate and improve the patient's eye condition, with particular reference to adjustment and/or modification frequency of drug administration.

In some embodiments, the method according to the present invention can comprise a step of determining a prediction of disease activity change as a result of a change from a current dosing regimen of the drug for treatment of the patient's eye disease to a different dosing regimen thereof. The disease activity change can be, for instance, disease progression or regression of w-AMD, or of another retinal disease, as above highlighted. In this case, the different treatment regimen preferably comprises a different drug administration frequency for treating the patient e.g. with the same dose of the drug as in the current or previous treatment regimen. For instance, given a current treatment regimen wherein a dose of X mL of the drug, like brolucizumab-dbII for treatment of w-AMD, is administered at a specific, first frequency, such as at 12 week intervals, the disease activity change is predicted for the case that the current treatment regimen is switched to a second dosing regimen with an administration frequency of 8 week intervals; or vice versa.

In various embodiments wherein a prediction of disease activity change is determined, the disease activity score can be linked to the probability, or to the appropriateness, of switching between different dosing regimens, or to a probabilistic prediction of disease activity change linked to the switching. Moreover, determining the prediction of disease activity change can be based on predicting a physiological change in one of the one or more identified anatomical variables over a period of time, for instance, in the amount of retinal thickness and/or volume loss or gain over a period of time; and/or in the rate of visual acuity loss or gain over a period of time; and/or in the rate of intraretinal fluid volume loss or gain over a period of time, when a change from the current, first dosing regimen to a second dosing regimen has been carried out.

In various embodiments wherein the dosing regimen comprises a drug administration frequency for treating the patient with a dose of the drug, the method according to the present invention can further comprise the step of generating, using a third algorithm, a drug administration frequency recommendation. The drug administration frequency recommendation can be based on the values of the one or more anatomical variables identified and/or on the disease activity score overall. The third algorithm can be substantially coincident with, or comprised in, the second algorithm; or it can be an additional algorithm different from the second algorithm.

In various embodiments, the drug administration frequency recommendation includes a parameter selected from the group consisting of:
a drug dosing frequency interval, e.g. how many weeks pass between subsequent injections;
a next drug dosing date;
a next drug dose amount;
a next retinal imaging date;
a next visit date for monitoring of the patient by a health care provider; and/or
a combination thereof.

Generating the drug administration frequency recommendation as above illustrated can, in some embodiments, comprise the steps of generating, using the third algorithm, one or more probabilistic simulations of treatment outcomes for different drug dosing regimens; and of eventually generating the drug administration frequency recommendation, based on such probabilistic simulations of treatment outcomes. The treatment outcomes simulated can be, for instance, visual acuity gain and/or treatment effect on the one or more identified anatomical variables, such as intraretinal fluid volume loss. These probabilistic simulation can take into account disease activity scores and/or drug administration frequency recommendations previously generated and output.

The third algorithm can also be used to make a prediction of time-dependent visual acuity gain, based on the generated drug administration frequency recommendation. Thus, it can be predicted how much the patient's visual acuity will improve in a given period of time, if treated according to the generated drug administration frequency recommendation.

In various embodiments, the set of input patient data, e.g. fed to the second algorithm, further comprises data selected from a group consisting of patient longitudinal data on visual acuity, e.g., current and previous Snellen chart measurements; patient longitudinal data on physiological characteristics, e.g., current and previous age, weight etc.; previous disease activity scores; previous drug administration frequency recommendations; and/or a combination thereof.

Preferably, the drug for treating the patient's eye disease according to the present invention is an anti-vascular endothelial growth factor drug, also designatable as anti-VEGF drug, including drugs having multiple modes of action wherein at least one of the modes is anti-VEGF (e.g., a bispecific antibody including anti-VEGF activity).

In some embodiments, the clinical trial data part of the historical data set used for training machine learning algorithms of the present method comprise data associated with one or more anti-VEGF drugs and effect thereof on at least one of the one or more identified anatomical variables.

In various embodiments, the patient's eye disease is one of:
wet age-related macular degeneration, also designatable as w-AMD;
diabetic retinopathy, also designatable as DR, including proliferative diabetic retinopathy (PDR) and non-proliferative diabetic retinopathy (NPDR); and/or
diabetic macular edema, also designatable as DME; and/or
myopic choroidal neovascularization, also designatable as mCNV; and/or
macular edema following retinal vein occlusion, also designatable as RVO, including Central Retinal Vein Occlusion (CRVO) and Branch Retinal Vein Occlusion (BRVO); and/or
retinopathy of prematurity, also designatable as ROP.

The present invention also relates to a system comprising a computing device including: one or more processors; one or more input and/or output elements; memory; and one or more programs stored in the memory. The one or more programs include instructions for executing the method above described.

Moreover, the present invention also relates to a non-transitory computer-readable storage medium which stores one or more programs configured to be executed by one or more processors of a computing system, wherein the one or more programs include instructions for executing the method above described.

In some embodiments, the method according to the present invention can be modified to apply one algorithm simultaneously to both the one or more retinal images and to non-image data components, in order to make an assessment of the level of activity of the disease in the at least one eye of the patient, and/or of the progression or regression of the disease with respect to a level of activity formerly determined for the patient. This case can particularly apply to a method employing one single neural network architecture where exact steps of segmenting retina structures or assessing disease activity or providing a dosing recommendation are not specified. In this instance, a deep learning function is not assigned to identify features predefined by software developers, but it is feature-agnostic and autonomously searches for features in order to ultimately achieve the target of quantifying disease activity and informing on optimal timing of medical interventions, including dosing frequency.

In the case of machine learning algorithms to produce an artificial intelligence engine for disease activity evaluation and dosing recommendation, when training e.g. the second machine learning algorithm by applying it to the historical set of patient data, the method according to the present invention can comprise the step of selectively using or not using the input variables comprised in the second machine learning algorithm, either in the entirety of the algorithm or in one or more steps of the algorithm.

Thus, each of the input variables contributes to the prediction to a different extent and is stored in the respective model. This can be characterized by an importance matrix wherein, to each of the variables actually used in the machine learning algorithms, is associated with a respective ‘importance’ value or weight, as resulting from a calculation adopting a given importance metric.

In various embodiments, the first algorithm and/or the second algorithm and/or the third algorithm are machine learning generated models comprising a gradient boosted decision trees algorithm, such as a LightGBM or an XGBoost algorithm; and/or an aggregation of decision trees, such as Bayesian Additive Regression Trees (BART); and/or a Recurrent Neural Network (RNN) algorithm.

XGBoost is a decision-tree-based ensemble machine learning algorithm that uses a gradient boosting framework. XGBoost is a parallelized tree learning algorithm. Each tree consists of a number of branches where the dataset is split corresponding to a chosen variable and a split value. After adding a split on this variable, two new branches are created. The number of splits is conventionally set to a smaller number (such as 3-10) which limits the ability of a single tree to fit a function accurately. But if many trees (ensembles) are combined, very accurate classifiers can be designed. In boosting algorithms, each tree aims to fit the instances better than it was executed by the previous trees.

LightGBM is also a gradient boosting framework that uses a tree based learning algorithm. Light GBM grows trees vertically while other algorithms grow trees horizontally, meaning that Light GBM grows trees leaf-wise while other algorithms grow level-wise. It will choose the leaf with max delta loss to grow. When growing the same leaf, a Leaf-wise algorithm can reduce more loss than a level-wise algorithm. The advantage of LightGBM is higher processing speed and focus on accuracy for larger data sets.

The Bayesian Additive Regression Tree (BART) model integrates multiple decision trees, where each tree is constrained by a regularization prior to be a weak learner, and fitting and inference are accomplished via an iterative Bayesian backfitting MCMC algorithm that generates samples from a posterior. Effectively, BART is a nonparametric Bayesian regression approach which uses dimensionally adaptive random basis elements. Similar to ensemble methods in general and boosting algorithms in particular, BART is defined by a statistical model integrating both a prior and a likelihood. This approach enables full posterior inference including point and interval estimates of the unknown regression function as well as the marginal effects of potential predictors.

Model hyperparameters can be optimized using an appropriate k-fold crossvalidation approach (e.g. splitting the data into k=4 parts and training 4 models, each time on ¾ of the data and evaluating the performance on the remaining %) while ensuring that data from the same patient is always either in a training or a validation fold.

The abovementioned gradient boosted decision tree algorithms, fitted for the assessment of disease activity level for a patient's eye, implemented by the method according to the present invention, effectively find the optimal split points among weighted input variables. The relative variable importance measures can be, by way of example, based on the number of times a variable is selected for splitting, weighted by the improvement to the model as a result of each split, and averaged over all trees.

In various embodiment of the method according to the present invention, the metric “gain” was employed to establish the relative variable importance. The gain can be defined as an attribute of the variables and it implies the relative contribution of the corresponding variable to the model, calculated by taking each variable's contribution for each tree in the model. A higher value of this metric for a given first variable, when compared to that of a different second variable, implies the first variable is more important for generating an assessment or prediction. “Gain” substantially is the improvement in accuracy brought by a variable to the tree branches it is on. In some embodiments, a gain is implemented as a Shapley value (see, for example Table 1). In some embodiments, a gain is implemented as other methods, e.g., SHapley Additive exPlanations (SHAP).

Recurrent neural networks (RNNs) are a class of artificial neural networks which use sequential data or time series data. They have internal memories that are able to capture all information stored in sequence, taking information from prior inputs to influence the current input and output. Thus, RNNs are powerful enough to make use of information in a relatively long sequence, since they can perform the same tasks for every single input of data in the sequence, with output of the current input dependent on previous computations.

### BRIEF DESCRIPTION OF THE DRAWINGS

The method and the system and according to the invention are described in more detail herein below by way of exemplary embodiments and with reference to the attached drawings, in which:

FIG. 1 illustrates an exemplary system for generating and utilizing a machine learning model that assesses a level of activity, including presence or an absence, of a disease causing neovascularization of a retina and/or in proximity of a retina of a patient's eye;

FIG. 2 illustrates an exemplary machine learning system in accordance with some embodiments;

FIG. 3 illustrates an exemplary electronic device in accordance with some embodiments;

FIG. 4 illustrates an exemplary process for training, validating, and testing one or more machine learning models for assessing the level of activity of the patient's eye disease;

FIG. 5 illustrates a high-level diagram of a flow of information from the patient, acquisition of a set of input patient data and subsequent elaboration of such data by an AI-based clinical decision support software implementing the method according to the present invention, outputting a treatment recommendation to a health care provider or physician;

FIG. 6 illustrates a representative portion of an exemplary historical data set for training the AI-based clinical decision support software of FIG. 5, including anonymized electronic health records for a given patient, relating to patient visits within a first 8-week loading phase and within a subsequent further 8-week treatment phase, leading up to a target 16th week wherein a first disease activity assessment is made by a masked investigator;

FIG. 7 shows a schematic representation of a first and a second approach of the AI-based clinical decision support software of FIG. 5;

FIG. 8 shows an example workflow for the second approach of the AI-based clinical decision support software of FIG. 7;

FIG. 9 illustrates a representative portion of an exemplary set of output data by the AI-based clinical decision support software of FIG. 5, the output data relating to a disease activity score at the target 16th week of treatment for corresponding 8 anonymized patients identified by numbers 2 to 9;

FIG. 10 illustrates a portion of a graphical user interface, e.g. at a health care professional's or ophthalmologist's front end, displaying data output by the AI-based clinical decision support software of FIG. 5, wherein the displayed data provides a rendition of current disease activity scores for a patient;

FIG. 11 illustrates a portion of a graphical user interface similar to that of FIG. 10, wherein the data displayed provides guidance on suitable drug dosing regimens for a patient examined, particularly showing how disease activity levels for the patient's eye can be predicted to change as a result of a change between two different dosing regimens of an anti-VEGF drug to be injected in the patient's eyes;

FIG. 12 illustrates a further portion of the graphical user interface of FIG. 11, wherein the relative importance of anatomical and functional features influencing the assessment of disease activity for the patient is rendered, as a further explanatory guide to the ophthalmologist;

FIG. 13 illustrates a further portion of the graphical user interface of FIG. 10, wherein the disease activity score for the examined patient is shown over time, across a loading phase and a subsequent maintenance phase of the treatment.

### DESCRIPTION OF EMBODIMENTS

In the following description certain terms are used for reasons of convenience and are not intended to limit the invention. The terms “right”, “left”, “up”, “down”, “under” and “above” refer to directions in the figures. The terminology comprises the explicitly mentioned terms as well as their derivations and terms with a similar meaning. Also, spatially relative terms, such as “beneath”, “below”, “lower”, “above”, “upper”, “proximal”, “distal”, and the like, may be used to describe one element's or feature's relationship to another element or feature as illustrated in the figures. These spatially relative terms are intended to encompass different positions and orientations of the devices in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. The devices may be otherwise oriented (rotated 90 degrees or at other orientations), and the spatially relative descriptors used herein interpreted accordingly.

Likewise, descriptions of movement along and around various axes include various special device positions and orientations.

To avoid repetition in the figures and the descriptions of the various aspects and illustrative embodiments, it should be understood that many features are common to many aspects and embodiments. Omission of an aspect from a description or figure does not imply that the aspect is missing from embodiments that incorporate that aspect. Instead, the aspect may have been omitted for clarity and to avoid prolix description. In this context, the following applies to the rest of this description: If, in order to clarify the drawings, a figure contains reference signs which are not explained in the directly associated part of the description, then it is referred to previous or following description sections. Further, for reason of lucidity, if in a drawing not all features of a part are provided with reference signs it is referred to other drawings showing the same part. Like numbers in two or more figures represent the same or similar elements.

The following description sets forth exemplary systems, devices, methods, parameters, and the like. It should be recognized, however, that such description is not intended as a limitation on the scope of the present disclosure but is instead provided as a description of exemplary embodiments. For example, reference is made to the accompanying drawings in which it is shown, by way of illustration, specific example embodiments. It is to be understood that changes can be made to such example embodiments without departing from the scope of the present disclosure.

As used herein, the term “subject” or “subjects” are equivalent to the term “patient” and refers to a mammalian organism, preferably a human being, who may be diagnosed with the condition (e.g., disease or disorder) of interest and who may benefit biologically, medically, or in quality of life from treatment for the condition.

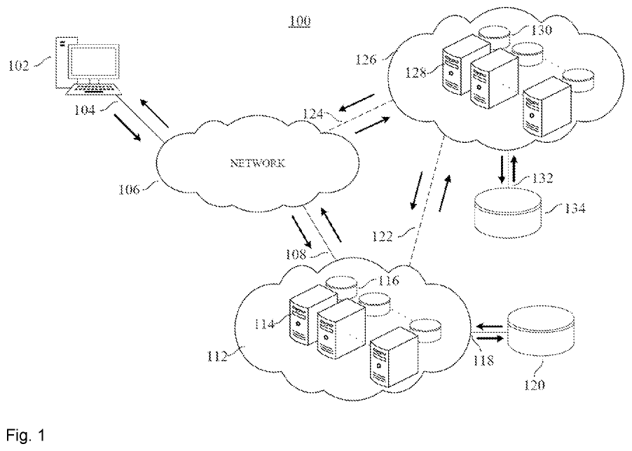

Attention is now directed to examples of electronic devices and systems for performing the techniques described herein in accordance with some embodiments. With reference to FIG. 1, an exemplary system 100 of electronic devices (e.g., such as electronic device 300) is shown. System 100 includes a client system 102. In some examples, client system 102 includes one or more electronic devices (e.g., 300). For example, client system 102 can represent a health care provider's (HCP) computing system (e.g., one or more computers in whatever form, such as mainframe, or personal, and can be used for the input, collection, and/or processing of subject data by a HCP, as well as for the output of subject data analysis (e.g., prognosis information). For further example, client system 102 can represent a subject's device (e.g., a home-use medical device; a personal electronic device such as a smartphone, tablet, desktop computer, or laptop computer) that is connected to one or more HCP electronic devices and/or to system 108, and that is used for the input and collection of subject data. In some examples, client system 102 includes one or more electronic devices (e.g., 300) networked together (e.g., via a local area network). In some examples, client system 102 includes a computer program or application (comprising instructions executable by one or more processors) for receiving subject data and/or communicating with one or more remote systems (e.g., 112, 126) for the processing of such subject data.

Client system 102 is connected to a network 106 via connection 104. Connection 104 can be used to transmit and/or receive data from one or more other electronic devices or systems (e.g., 112, 126). The network 106 may include any type of network that allows sending and receiving communication signals, such as a wireless telecommunication network, a cellular telephone network, a time division multiple access (TDMA) network, a code division multiple access (CDMA) network, Global System for Mobile communications (GSM), a third-generation (3G) network, fourth-generation (4G) network, fifth-generation (5G) network, a satellite communications network, and other communication networks. The network 106 may include one or more of a Wide Area Network (WAN) (e.g., the Internet), a Local Area Network (LAN), and a Personal Area Network (PAN). In some examples, the network 106 includes a combination of data networks, telecommunication networks, and a combination of data and telecommunication networks. The systems and resources 102, 112 and/or 126 communicate with each other by sending and receiving signals (wired or wireless) via the network 106. In some examples, the network 106 provides access to cloud computing resources (e.g., system 112), which may be elastic/on-demand computing and/or storage resources available over the network 106. The term ‘cloud’ services generally refers to a service performed not locally on a user's device, but rather delivered from one or more remote devices accessible via one or more networks.

Cloud computing system 112 is connected to network 106 via connection 108. Connection 108 can be used to transmit and/or receive data from one or more other electronic devices or systems and can be any suitable type of data connection (e.g., wired, wireless, or any combination of wired and wireless). In some examples, cloud computing system 112 is a distributed system (e.g., remote environment) having scalable/elastic computing resources. In some examples, computing resources include one or more computing resources 114 (e.g., data processing hardware). In some examples, such resources include one or more storage resources 116 (e.g., memory hardware). The cloud computing system 112 can perform processing (e.g., applying one or more machine learning models, applying one or more algorithms) of subject data (e.g., received from client system 102). In some examples, cloud computing system 112 hosts a service (e.g., computer program or application comprising instructions executable by one or more processors) for receiving and processing subject data (e.g., from one or more remote client systems, such as 102). In this way, cloud computing system 112 can provide subject data analysis services to a plurality of health care providers (e.g., via network 106). The service can provide a client system 102 with, or otherwise make available, a client application (e.g., a mobile application, a web-site application, or a downloadable program that includes a set of instructions) executable on client system 102. In some examples, a client system (e.g., 102) communicates with a server-side application (e.g., the service) on a cloud computing system (e.g., 112) using an application programming interface.

In some examples, cloud computing system 112 includes a database 120. In some examples, database 120 is external to (e.g., remote from) cloud computing system 112. In some examples, database 120 is used for storing one or more of subject data, algorithms, machine learning models, or any other information used by cloud computing system 112.

In some examples, system 100 includes cloud computing resource 126. In some examples, cloud computing resource 126 provides external data processing and/or data storage service to cloud computing system 112. For example, cloud computing resource 126 can perform resource-intensive processing tasks, such as machine learning model training, as directed by the cloud computing system 112. In some examples, cloud computing resource 126 is connected to network 106 via connection 124. Connection 124 can be used to transmit and/or receive data from one or more other electronic devices or systems and can be any suitable type of data connection (e.g., wired, wireless, or any combination of wired and wireless). For example, cloud computing system 112 and cloud computing resource 126 can communicate via network 106, and connections 108 and 124. In some examples, cloud computing resource 126 is connected to cloud computing system 112 via connection 122. Connection 122 can be used to transmit and/or receive data from one or more other electronic devices or systems and can be any suitable type of data connection (e.g., wired, wireless, or any combination of wired and wireless). For example, cloud computing system 112 and cloud computing resource 126 can communicate via connection 122, which is a private connection.

In some examples, cloud computing resource 126 is a distributed system (e.g., remote environment) having scalable/elastic computing resources. In some examples, computing resources include one or more computing resources 128 (e.g., data processing hardware). In some examples, such resources include one or more storage resources 130 (e.g., memory hardware). The cloud computing resource 126 can perform processing (e.g., applying one or more machine learning models, applying one or more algorithms) of subject data (e.g., received from client system 102 or cloud computing system 112). In some examples, cloud computing system (e.g., 112) communicates with a cloud computing resource (e.g., 126) using an application programming interface.

In some examples, cloud computing resource 126 includes a database 134. In some examples, database 134 is external to (e.g., remote from) cloud computing resource 126. In some examples, database 134 is used for storing one or more of subject data, algorithms, machine learning models, or any other information used by cloud computing resource 126.

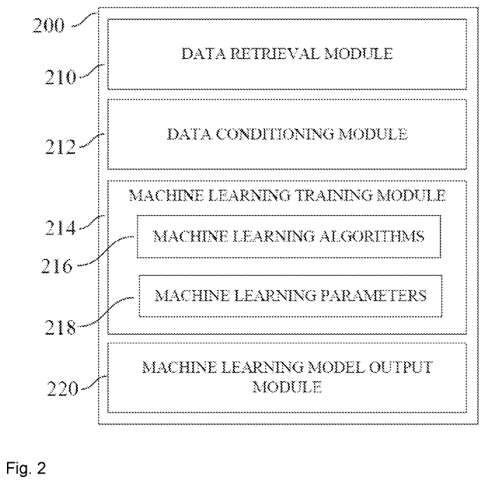

FIG. 2 illustrates an exemplary machine learning system 200 in accordance with some embodiments. In some embodiments, a machine learning system (e.g., 200) is comprised of one or more electronic devices (e.g., 300). In some embodiments, a machine learning system includes one or more modules for performing tasks related to one or more of training one or more machine learning algorithms, applying one or more machine learning models, and outputting and/or manipulating results of machine learning model output. Machine learning system 200 includes several exemplary modules. In some embodiments, a module is implemented in hardware (e.g., a dedicated circuit), in software (e.g., a computer program comprising instructions executed by one or more processors), or some combination of both hardware and software. In some embodiments, the functions described below with respect to the modules of machine learning system 200 are performed by two or more electronic devices that are connected locally, remotely, or some combination of both. For example, the functions described below with respect to the modules of machine learning system 200 can be performed by electronic devices located remotely from each other (e.g., a device within system 112 performs data conditioning, and a device within system 126 performs machine learning training).

In some embodiments, machine learning system 200 includes a data retrieval module 210. Data retrieval module 210 can provide functionality related to acquiring and/or receiving input data for processing using machine learning algorithms and/or machine learning models. For example, data retrieval module 210 can interface with a client system (e.g., 102) or server system (e.g., 112) to receive data that will be processed, including establishing communication and managing transfer of data via one or more communication protocols.

In some embodiments, machine learning system 200 includes a data conditioning module 212. Data conditioning module 212 can provide functionality related to preparing input data for processing. For example, data conditioning can include making a plurality of images uniform in size (e.g., cropping, resizing), augmenting data (e.g., taking a single image and creating slightly different variations (e.g., by pixel rescaling, shear, zoom, rotating/flipping), extrapolating, variable engineering, filtering and/or cleaning data, mapping and structuring of variables retrieved from databases, merging data sets from different sources or clinical study sites, segregating data by patient, constructing an observational research file or the like.

In some embodiments, machine learning system 200 includes a machine learning training module 214. Machine learning training module 214 can provide functionality related to training one or more machine learning algorithms, in order to create one or more trained machine learning models.

The concept of “machine learning” generally refers to the use of one or more electronic devices to perform one or more tasks without being explicitly programmed to perform such tasks. A machine learning algorithm can be “trained” to perform the one or more tasks (e.g., classify an input data into one or more classes, identify and classify variables within input data, predict a value based on input data) by applying the algorithm to a set of training data, in order to create a “machine learning model” (e.g., which can be applied to non-training data to perform the tasks). A “machine learning model” (also referred to herein as a “machine learning model artefact” or “machine learning artefact”) refers to an artefact that is created by the process of training a machine learning algorithm. The machine learning model can be a mathematical representation (e.g., a mathematical expression) to which an input can be applied to get an output. As referred to herein, “applying” a machine learning model can refer to using the machine learning model to process input data (e.g., performing mathematical computations using the input data) to obtain some output.

Training of a machine learning algorithm can comprise “supervised” or “unsupervised” learning. Generally speaking, a supervised machine learning algorithm builds a machine learning model by processing training data that includes both input data and desired outputs (e.g., for each input data, the correct answer (also referred to as the “target” or “target attribute”) to the processing task that the machine learning model is to perform). Supervised training is useful for developing a model that will be used to make predictions based on input data. An unsupervised machine learning algorithm builds a machine learning model by processing training data that only includes input data (no outputs). Unsupervised training is useful for determining structure within input data. Alternatively, semi-supervised and/or reinforcement machine learning algorithms can also be employed. A combination of all or some of the abovementioned machine learning algorithms to carry out the method according to the present invention is also envisaged. A machine learning algorithm can be implemented using a variety of techniques, including the use of one or more of a gradient boosted tree, an artificial neural network, a deep neural network, transformers or long short-term memory recurrent neural networks and the like.

Referring again to FIG. 2, in some examples, machine learning training module 214 includes one or more machine learning algorithms 216 that will be trained. In some examples, machine learning training module 214 includes one or more machine learning parameters 218. For example, training a machine learning algorithm can involve using one or more parameters 218 that can be defined (e.g., by a user, or by hyper parameter optimization) that affect the performance of the resulting machine learning model. Machine learning system 200 can receive (e.g., via user input at an electronic device) and store such parameters for use during training. Exemplary parameters include stride, pooling layer settings, kernel size, number of filters, learning rate, maximum tree depth, subsample ratios, and the like, however this list is not intended to be exhaustive.

In some examples, machine learning system 200 includes machine learning model output module 220. Machine learning model output module 220 can provide functionality related to outputting a machine learning model, for example, based on the processing of training data. Outputting a machine learning model can include transmitting a machine learning model to one or more remote devices. For example, a machine learning system 200 implemented on electronic devices of cloud computing resource 126 can transmit a machine learning model to cloud computing system 112, for use in processing subject data sent between client system 102 and system 112.

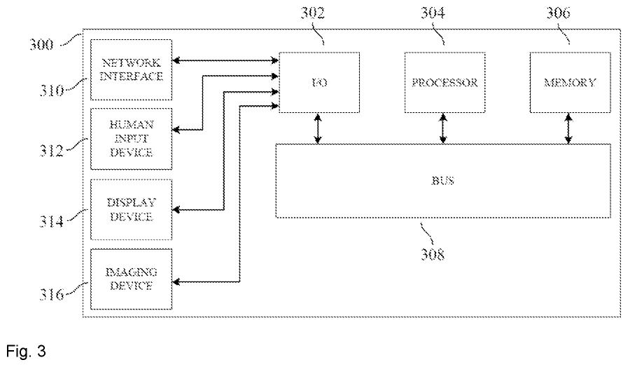

FIG. 3 illustrates exemplary electronic device 300 which can be used in accordance with some examples. Electronic device 300 can represent, for example, a PC, a smartphone, a server, a workstation computer, a medical device, or the like. In some examples, electronic device 300 comprises a bus 308 that connects input/output (I/O) section 302, one or more processors 304, and memory 306. In some examples, electronic device 300 includes one or more network interface devices 310 (e.g., a network interface card, an antenna). In some examples, I/O section 302 is connected to the one or more network interface devices 310. In some examples, electronic device 300 includes one or more human input devices 312 (e.g., keyboard, mouse, touch-sensitive surface). In some examples, I/O section 302 is connected to the one or more human input devices 312. In some examples, electronic device 300 includes one or more display devices 314 (e.g., a computer monitor, a liquid crystal display (LCD), light-emitting diode (LED) display). In some examples, I/O section 302 is connected to the one or more display devices 314. In some examples, I/O section 302 is connected to one or more external display devices. In some examples, electronic device 300 includes one or more imaging device 316 (e.g., a camera, a device for capturing medical images). In some examples, I/O section 302 is connected to the imaging device 316 (e.g., a device that includes a computer-readable medium, a device that interfaces with a computer readable medium).

In some examples, memory 306 includes one or more computer-readable media that store (e.g., tangibly embody) one or more computer programs (e.g., including computer executable instructions) and/or data for performing techniques described herein in accordance with some examples. In some examples, the computer-readable medium of memory 306 is a non-transitory computer-readable medium. At least some values based on the results of the techniques described herein can be saved into memory, such as memory 306, for subsequent use. In some examples, a computer program is downloaded into memory 306 as a software application. In some examples, one or more processors 304 include one or more application-specific chipsets for carrying out the above-described techniques.

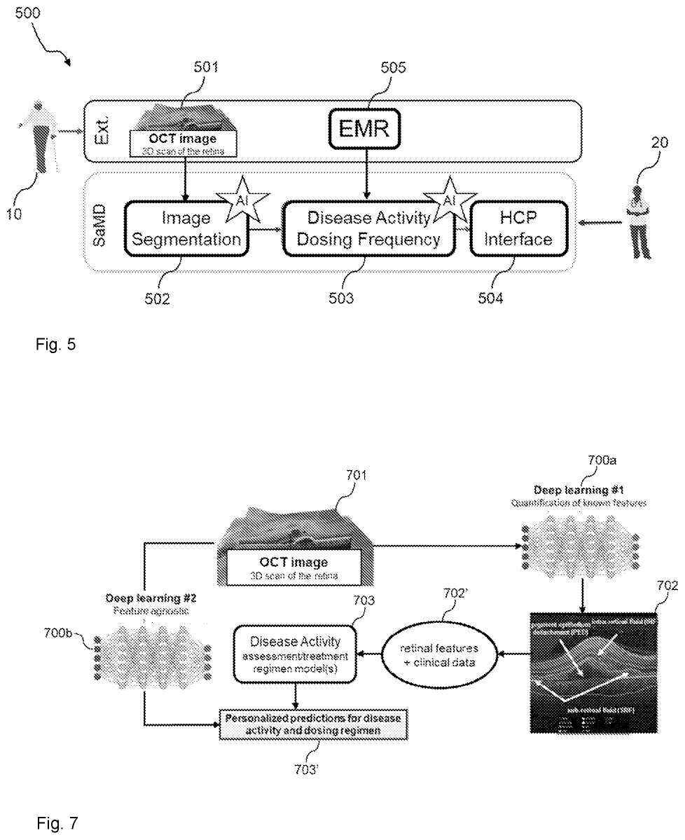

FIG. 5 shows a high-level diagram of an artificial intelligence-, or AI-, based clinical decision support software, implementing embodiments of the method according to the present invention. In FIG. 5 it is illustrated the flow of information, from the input relating to a given patient to a customized clinical decision support recommendation output to a health care professional. Such AI-based clinical decision support software comprises several AI models or artifacts, namely an image segmentation model, a disease activity assessment model, as well as a dosing frequency recommendation model, based on machine learning algorithms.

Ultimately, the AI-based clinical decision support software contributes to assess the level of disease activity in the patient's eye and provides personalized dosing frequency recommendations to the health care provider, based on features automatically extracted from OCT images and other non-imaging patient features of the patient.

As already explained, the disease activity assessment model is generated by one or more machine learning algorithms comprising a multiplicity of input variables. The input variables correspond to anatomical variables, as identified from the OCT images, and to distinct clinical, non-image derived input patient data.

The one or more machine learning algorithms are trained on a historical set of patient data from a plurality of historical patients diagnosed with an eye, or ocular, disease.

While the method of the present invention will be in the following explained with respect to patients affected by neovascular age-related macular degeneration (w-AMD, or nAMD), mutatis mutandis the method can be applied to assess the disease activity of patients affected by an ocular disease selected from a list consisting of: abnormal angiogenesis, choroidal neovascularization (CNV), retinal vascular permeability, retinal edema, diabetic retinopathy (particularly proliferative diabetic retinopathy (PDR) and non-proliferative diabetic retinopathy (NPDR)), macular edema (ME), diabetic macular edema (DME), neovascular (exudative) age-related macular degeneration (nAMD), choroidal neovascularization (CNV) associated with nAMD, sequela associated with retinal ischemia, Retinal Vein Occlusion (RVO), Central Retinal Vein Occlusion (CRVO), Branch Retinal Vein Occlusion (BRVO), macular edema following retinal vein occlusion, and posterior segment neovascularization.

An exemplary disease referred to in the following embodiments is neovascular age-related macular degeneration, particularly w-AMD; and an exemplary drug for treatment is an anti-vascular endothelial growth factor drug, also designatable as anti-VEGF drug, such as brolucizumab, also known as RTH258 and commercially as Beovu®.

The personalized dosing frequency recommendations to the health care provider obtained by the AI-based clinical decision support software of the present invention are at any rate comprised in the range of approved dosing frequencies, as stated in the brolucizumab labeling for treatment of w-AMD. This corresponds to a regimen of: by intravitreal injection monthly (approximately every 25-31 days) for the first three doses, followed by 6 mg (0.05 mL) by intravitreal injection once every 8-12 weeks. Thanks to the method according to the present invention, a dosing frequency recommendation can be determined as early as after the first 16 weeks of treatment (i.e. 8 weeks following the end of the 8-week loading phase).

The historical set of patient data includes input values for the identified anatomical variables, derived from retinal images of the historical patients. The historical set of patient data also includes values correlated to clinical, non-image derived input patient data, comprising historical patients' demographics and medical history.

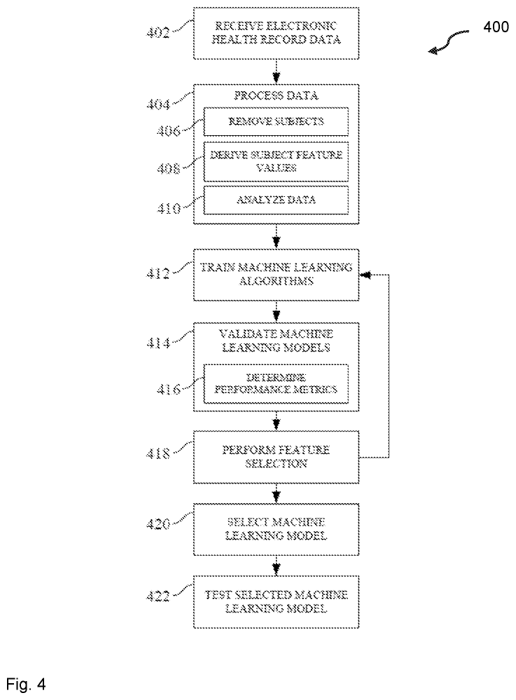

FIG. 4 illustrates an exemplary process for training, validating, and testing one or more machine learning models for assessing a level of activity, including presence or an absence, of a neovascular ocular disease.

Process 400 is merely exemplary. Thus, some operations in method 400 are, optionally, combined, the orders of some operations are, optionally, changed, and some operations are, optionally, omitted. In some examples, process 400 is performed by a system having one or more features of system 100, shown in FIG. 1, and/or system 200, shown in FIG. 2. For example, one or more blocks of process 400 can be performed by client system 102, cloud computing system 112, and/or cloud computing resource 126.

Process 400 is described with reference to an exemplary application which uses, as historical set of patient data for model development, real-world data collected in the course of two completed Novartis clinical trials. In these two pivotal Phase 3 Novartis studies called HAWK and HARRIER, the efficacy and safety of brolucizumab in patients with nAMD, or w-AMD, has been tested.

HAWK and HARRIER are two-year, randomized, double-masked studies to evaluate the efficacy and safety of intravitreal injections of brolucizumab 6 mg (HAWK and HARRIER) and brolucizumab 3 mg (HAWK only) versus aflibercept 2 mg in patients with nAMD. Respectively, 1082 and 743 patients were enrolled for carrying out the HAWK and HARRIER clinical studies. Reference will be made to data collected in the Phase 3 of these clinical trials, also respectively known by ClinicalTrials.gov identifier numbers NCT02307682 and NCT02434328.

In context of HAWK and HARRIER clinical trials, disease activity (DA) assessment is the step in which physicians determine which brolucizumab patients are suitable for a 12-week dosing interval and which patients instead should be adjusted to an 8-week interval. In all patients, regardless of treatment arm, disease activity was assessed by a masked investigator. Among patients who received brolucizumab, if the masked investigator determined disease activity to be present, dosing intervals were adjusted to q8w dosing and they remained at q8w, or 8-week dosing intervals, for the remainder of the study.

In the HAWK and HARRIER trials, the severity of nAMD, or w-AMD, was assessed by measuring a multiplicity of key variables, as already mentioned above. An example of these variables comprises at least:
best corrected visual acuity (BCVA), measuring the best vision one can achieve with correction (such as glasses), using a standard visual acuity testing chart called the ETDRS chart (Early Treatment Diabetic Retinopathy Study chart);
central subfield thickness (CST, or CSFT), wherein increases in CST may indicate abnormal fluid accumulation (known as macular edema) in the fovea, that is the part of the retina responsible for sharp, central vision;
sub retinal fluid (SRF) and intra-retinal fluid (IRF), indicating an accumulation of abnormal fluid pockets that may damage cells and surrounding tissue; and
sub-retinal pigment epithelium (RPE) fluid, that is an accumulation of fluid under the sub-retinal pigment epithelium which may cause a reduction in visual acuity.

How the former and further variables contribute to the development of the disease assessment model implementing the method of the present invention will be clarified in the following.

Data of randomized and treated patients from the HAWK and HARRIER clinical trials were included for model training. Therefore, a total of 1817 patients treated during the Hawk & Harrier clinical trials were inserted in the plurality of patients whose data builds up the historical set of patient data. Eligibility was defined by the inclusion and exclusion criteria of the respective, published clinical trial protocols.

The available data from HAWK and HARRIER is partitioned into two non-overlapping subsets: a training set for model development and selection (80% of the data), and a validation set (20% of data) withheld for testing the final model performance.

The machine learning algorithms used to ultimately obtain the disease assessment model of the present invention can, in embodiments of the invention, be additionally refined by an expert adjudication process comprising using treatment decisions made by masked evaluating investigators to further train the model. In this case, the automatically output disease activity assessments can be reviewed by an independent panel composed of retina specialists experienced in w-AMD treatment and retina imaging. Adjudication cases can therefore be selected and reviewed in multiple steps, following an iterative process enabling adjustment of case selection and distribution among panelists, dynamically using an updated model retrained on collected disease activity assessments by the adjudication panelists during the previous iterations. Thus, for each iteration of the adjudication process, the case selection can be based on a disease activity assessment model which has been further trained with the data generated during the previous iterations.

At block 402, a computing system (e.g., client system 102, cloud computing system 112, and/or cloud computing resource 126; electronic device 300) receives a data set (e.g., via data retrieval module 210) including electronic health records related to eye health from an external source (e.g., database 120 or database 134). The data set includes values of several anatomical variables, identified thanks to segmentation of retinal images of a plurality of patients having a confirmed diagnosis of w-AMD, as exemplified in FIG. 5; and values of clinical, non-image derived input patient data, such as longitudinal pseudonymized electronic health records for the same patients.

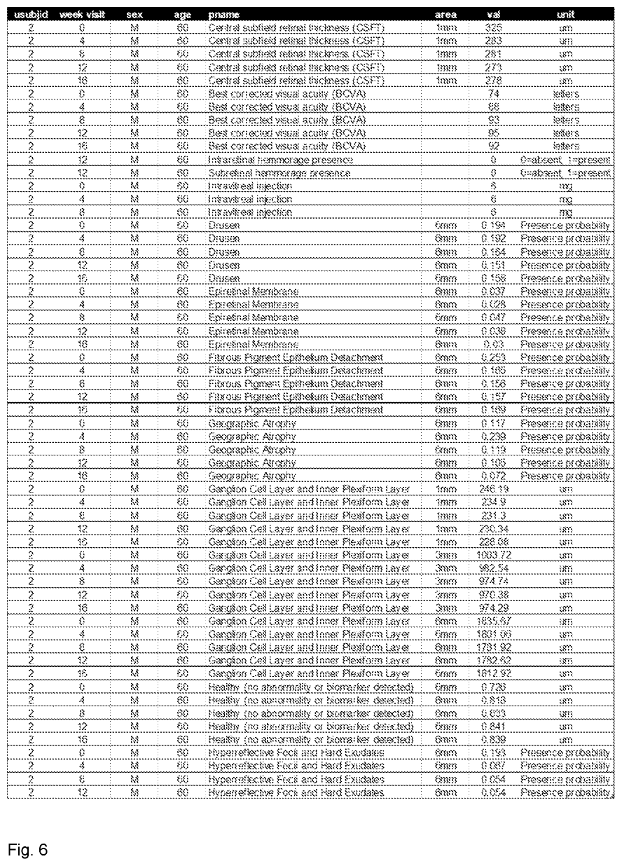

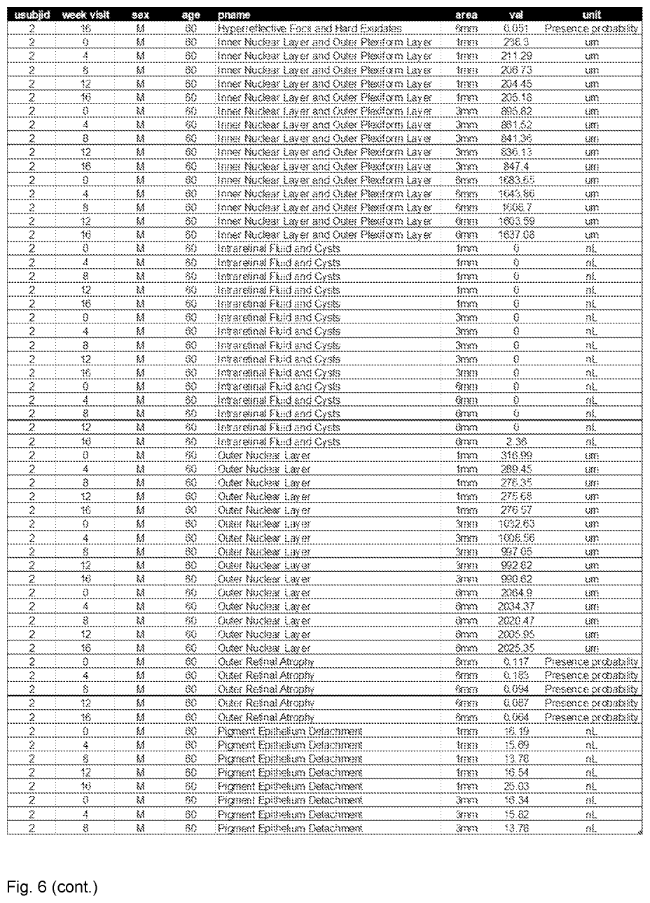

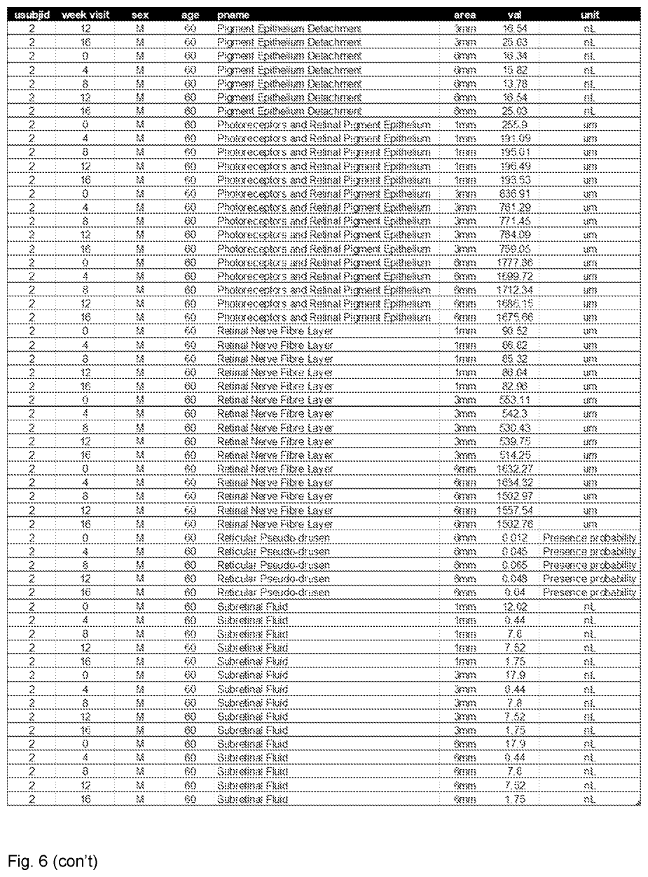

FIG. 6 shows an example of anonymized electronic health records for a given patient, or subject, indicated as “2”, comprised in an exemplary historical data set, relative to his visits within a first 8-week loading phase and within a subsequent further 8-week treatment phase, leading up to a target 16^{th} week, at which point a first disease activity assessment is made by a masked investigator. In some cases, the variable values are quantified in the form of presence probability or are attributed binary presence/absence coefficients.

In some examples, the computing system receives more than one historical data set including anonymized electronic health records related to a retinopathy from one or more sources. In some examples, block 402 further includes the computing system combining multiple received historical data sets into a single combined historical data set.

In the specific study carried out to develop one embodiment of the present invention, all patient-level data handled were pseudonymized without any identification of patient identity possible.

In some examples, the historical data set received at block 402 includes a higher number of values for a respective higher number of input variables than those included in exemplary data set, for a given subject.

In some examples, the data set received at block 402 includes less data inputs than those included in exemplary data set, for a given subject. It should be understood that the above list of subject variable, or features, is not exhaustive and that, in some examples, the computing system also receives descriptive data for one or more subjects of the plurality of subjects included in the data set received at block 402 (e.g., other subject diagnoses, subject medications, etc.).

Returning to FIG. 4, at block 404, the computing system processes (e.g., via data conditioning module 212) the data set received at block 402. In the examples mentioned above where the computing system receives more than one data set at block 402, the computing system processes the single combined data set. As shown in FIG. 4, processing the data set at block 404 may comprise the computing system excluding subjects from the data set at block 406, deriving subject feature values for the data set at block 408, and analyzing subject data included in the data set at block 410.

At block 406, in some embodiments the computing system removes one or more subjects from the data set based on a predetermined set of inclusion and/or exclusion criteria. For instance, a subject missing one or more scheduled visits may be discarded from the analysis; a subject missing one or more measurements in his/her history may be discarded. In some embodiments, the computing system does not remove any subjects.

At block 408, the computing system derives patient input variable values for the plurality of variables included in the data set. The computing system derives input values for input variables corresponding to a patient which are included in the historical data set for that subject.

In some examples, the computing system derives subject variable or feature values for one or more subjects based on previous (e.g., older) values for the subject feature (e.g., using a time window methodology). For instance, a supervised machine learning algorithm can implement a pre-processing step of calculating the values of the multiplicity of input variables as recorded at preset time periods before the assessment date, such as 0, 4, 8, 12 weeks before the assessment date. In some cases, a linear interpolation or extrapolation may be used; spline based methods may be used.

If a patient does not have any measurement before the assessment date, this patient is kept and the periods without measurement can be treated as NULL. Patients without any measurements can also be kept and have NULL for all their assessment dates.

Referring to FIGS. 4 at block 410, and 2 at block 212, the computing system analyzes the subject data included in the data set. Specifically, the computing system analyzes input variable values, or subject feature values, e.g., using one or more statistical tests and/or techniques, for one or more patients included in the data set at block 410. This step is carried out to determine statistical associations between the one or more input variables and disease activity (e.g., to determine if individual input variables have explanatory power to differentiate patients that will have higher disease activity from patients that have lower disease activity). This step can be especially suitable and advantageous in case of implementing an XGBoost machine learning algorithm.

In some examples, block 404 does not include one of block 406, block 408, and block 410.

In some examples, processing the data set at block 404 further includes the computing system removing repeated, nonsensical, or unnecessary subject features (and their corresponding values) from the data set and/or aligning units of measurement for subject feature values included in the data set.

In some examples, processing the data set at block 404 further includes the computing system one-hot encoding, or creating embeddings, for categorical (i.e. non numeric) subject feature values for one or more subjects of the plurality of subjects included in the data set.

At block 412, the computing system trains a plurality of machine learning algorithms (e.g., included in machine learning algorithms 216) by separately applying each of the plurality of machine learning algorithms (e.g., via machine learning training module 214) to patient historical data included in the processed data set. For example, the computing system separately applies a plurality of supervised machine learning algorithms including (but are not limited to) a logistic regression algorithm; a gradient boosted trees algorithm, such as a LightGBM or an XGBoost algorithm; a Recurrent Neural Network (RNN); and a random forest algorithm. In some examples, the computing system may apply one or more unsupervised or semi-supervised machine learning algorithms to subject data included in the processed data set.

In some embodiments wherein tree-based algorithms are trained, the computing system starts with identifying features as one or more root variables which can produce good predictive performance, followed by searching for additional layer of leaf variables whose attachment to the root variables can increase the predictive performance; the search is recursively implemented (i.e., by seeking a new layer of leaf variables whose addition to a previous layer of leaf variables can improve the predictive performance) until the predictive performance cannot be improved. In some cases, a learned tree-based algorithm may adapt its tree structure when one or more new samples are seen by the algorithm.

In some embodiments wherein RNN-based algorithms are trained, the computing system comprises a neural network able to analyze longitudinal data. The neural network can transform input data into a predictive score and a set of agnostic features. The agnostic features are further fed back to the neural network as part of the input for the next time point; that means, the input for the next time point includes patients' data and the agnostic features derived from the previous time point. Mathematically, the agnostic features capture important information in the history that can help the algorithms to make better prediction in the future. The neural network is trained until the predictive performance cannot improved.

Applying the machine learning algorithms to patient data included in the processed data set includes the computing system dividing the processed data set into a first portion (referred to herein as a “training set”) and a second portion (referred to herein as a “validation set”). In some embodiments, the computing system further divides the processed data set into a third portion (referred to herein as a “test set”). The training set is used to train the machine learning algorithms and generate machine learning models based on the training. The validation set is used to evaluate the generated machined learning models as well as update machine learning model hyperparameters for better performance. Instead of a single validation set, cross validation may be used. I.e. multiple training and validation data sets may be randomly created, in which each record is in a validation set once. Cross-validation (CV) may be implemented in such a way that records from a specific patient are always all in the same training or validation set to avoid information leakage. The test set is used to assess how well the trained machine learning models perform on unseen data, and is also used to estimate machine learning model performance when applied to new sets of subject data (e.g., subject data that is not included in the processed data set).

For example, in one preferred embodiment disclosed in the following, as already mentioned, 80% of the data of the historical data set is used as a training set for model development and selection, while 20% of the data serves as a validation set. However, the computing system can divide the processed data set alternatively, allocating different percentages of the subjects included in the processed data set (and of their corresponding subject feature values) as the training set, or validation set, or as test set. In various embodiments, the percentages may be 100% versus 0%, 95% versus 5%, 90% versus 10%, 85% versus 15%, or 75% versus 25%.

If, for instance, a supervised machine learning technique is employed, in some embodiments, applying machine learning algorithms to subject data included in the processed data set further includes the computing system labelling the disease activity level as a target attribute and subsequently training the machine learning algorithms using the training set. As discussed above, a target attribute represents the “correct answer” that a machine learning algorithm is trained to predict. Thus, in this case, each of the plurality of machine learning algorithms is trained using the training set (e.g., the subject feature values of the training set) so that the machine learning algorithms learn to output a disease activity linked to the probability, or the appropriateness, of effectively switching from a current dosing regimen for the drug used for treatment of the patient's eye disease, to a different dosing regimen, when provided with data similar to the training set (e.g., subject data including a plurality of subject features).

After separately training the plurality of machine learning algorithms, the computing system generates a machine learning model (e.g., via machine learning model output module 220) corresponding to each machine learning algorithm that is trained. For example, the computing system separately generates a machine learning model corresponding to a trained logistic regression algorithm, a machine learning model corresponding to a trained XGBoost algorithm, a machine learning model corresponding to a trained LightGBM algorithm, and a machine learning model corresponding to a trained random forest algorithm. Generating the machine learning models includes the computing system determining, based on the training of the machine learning algorithms, one or more patterns that map the values of the subject features included in the training set to the patients' corresponding disease activity level (e.g., the target attribute). Thereafter, the computing system generates the machine learning models representing the one or more patterns. The computing system uses a generated machine learning model (e.g., of the plurality of generated machine learning models) to output a disease activity score and/or predict a disease activity change as a result of a change between dosing regimens, when provided with data similar to the training set (e.g., specific patient data including input values for one or more of the patient input variables included in the training set).

At block 414, the computing system validates the machine learning models generated at block 412 using the validation set of the processed data set. Validating a machine learning model assesses the machine learning model's ability to accurately assess or predict a target attribute (in this case, a disease activity level, particularly in the case of w-AMD) when provided with data similar to the data used to train the machine learning algorithm that generated the machine learning model. As shown in FIG. 4, validating the machine learning models at block 414 includes the computing system determining performance metrics at block 416.

At block 416, the computing system determines one or more performance metrics for one or more of the machine learning models generated at block 412. For example, the computing system determines one or more performance metrics for each of the logistic regression machine learning model, XGBoost machine learning model, Light GBM machine learning model, and random forest machine learning model. The one or more performance metrics include (but are not limited to) recall, precision, area under the recall-precision curve (AuPRC); area under receiver operating characteristic curve (AuROC).

Recall, otherwise known as sensitivity, equals the proportion of events correctly predicted; precision, otherwise known as positive predictive value, equals the proportion of predicted events that actually happen; AuPRC equals the area under precision-recall curve and measures the performance across different thresholds for making a prediction. An AuROC curve is a graph plotting two parameters, True Positive Rate and False Positive, signifying the probability that a model ranks a random positive example more highly than a random negative example and showing the performance of a classification model across all possible classification thresholds.

At block 418, the computing system performs feature selection based on the machine learning models generated at block 412 and subject variables, or features, included in the processed data set. This step is separately performed for each of the machine learning models generated at block 412. The computing system preferably determines a performance metric for each subject variable or feature included in the training set and/or validation set. Specifically, the computing system may preferably use the gain metrics to narrow down the most important subject features (e.g., included in the training set and/or in the validation set) with respect to accurately and reliably assessing a disease activity level in retinopathies, particularly in the case of w-AMD. Alternatively, to this purpose, a Recursive Feature Elimination (RFE) can be employed, that is an iterative feature selection technique, e.g. determining an AUC performance metric for each subject feature. Based on the determined performance metrics, the computing system determines each subject feature's relative importance percentage. Then, the computing system removes the least important subject feature for each machine learning model (based on the determined relative importance percentages). As a result, a reduced training sets and a reduced validation sets are produced which help to avoid overfitting a machine learning model (as less redundant data is present after each of a succession of rounds of feature selection), to increase prediction performance, and to reduce the amount of time needed for a machine learning model to generate a prediction.

After the computing system generates the reduced training sets and the reduced validation sets, the computing system repeats the actions performed at blocks 412-418 using the reduced training sets and the reduced validation sets (instead of the original training set/validation set that included all subject features) for each of the same machine learning algorithms that were previously used (e.g., the logistic regression algorithm, XGBoost algorithm and random forest algorithm). Further, for each iteration of blocks 412-418, the computing system determines a performance metric for each subject feature included in the reduced training sets/reduced validation sets (e.g., at block 418) so that the computing system can once again determine and remove the least important subject feature included in the reduced training sets/reduced validation sets. By way of example, in the study that resulted in data for embodiments of the present invention, when segmentation outputs were employed, more than 100 input variables or features were originally created to be explored, in order to identify factors deemed as biomarkers or predictors of w-AMD; while, when raw image data are used as input (i.e., without segmentation step), more than 2,500,000 input variables or features were originally created to be explored for biomarkers identification.

The computing system will iteratively perform the actions of blocks 412-418, determining performance metrics at block 416 based on machine learning models that are trained/validated using reduced training sets/reduced validation sets. In this manner, the computing system (1) generates a plurality of machine learning models for each of the machine learning algorithms being used, (2) determines one or more performance metrics for each of the plurality of generated machine learning models, and (3) determines a relative feature importance percentage for each subject feature used to train each machine learning model.

At block 420, the computing system selects a machine learning model and selected set of features based on the results on the validation set or from cross-validation. Specifically, the computing system selects a machine learning model based on the one or more performance metrics determined for the various models generated during recursive feature elimination. In this case, the computing system selects the machine learning model (of all of the machine learning models generated for each of the machine learning algorithms used) that has the highest performance, in consideration of the primary objective on the validation set or based on cross-validation.

At block 422, the computing system tests the selected machine learning model using a data set of unseen subject data. Testing the selected machine learning model at block 422 includes the computing system determining one or more performance metrics based on the application of the selected machine learning model to the data set of unseen subject data. In the examples where the computing system further divides the processed data into training, validation and test sets (e.g., at block 412), the selected machine learning model is tested using the test set.

With reference to FIG. 7, two possible approaches are presented for the generation of respective AI-driven models 700a, 700b for assessing a level of activity, including presence or an absence, of a retinopathy in a patient's eye, namely w-AMD in this specific case. The models 700a, 700b are more precisely based on Recurrent Neural Networks (RNNs), that is, a class of neural networks that allow previous outputs to be used as inputs while having hidden states. The models 700a, 700b ultimately allow personalized assessment of disease activity and, accordingly, recommendation of optimal dosing regimen, as shown at block 703′.

The path in FIG. 7 designated by “Deep Learning #1” is substantially feature-based. A deep learning algorithm 700a is first applied to raw OCT images 701 to detect and/or to consequently measure relevant anatomical variables, or features, such as biomarkers of intraretinal fluid (IRF), Subretinal fluid (SRF), and Pigment Epithelial Detachment (PED). Detection and volume quantification is carried out by AI-based segmentation of the raw OCT images 701. Given an image 701, a segmenting neural network identifies the boundaries of detailed retina structures and measure their dimensions (e.g., thicknesses, areas or volumes), as shown at block 702. More extensive examples of structural information include: central subfield foveal thickness (CSFT); hyperreflective focii, ganglion cell layer and inner plexiform layer; inner nuclear layer and outer plexiform layer volume; intraretinal fluid and cysts volume; outer nuclear layer volume; pigment epithelium detachment volume; photoreceptors and retinal pigment volume; retinal nerve fiber layer volume; subretinal fluid volume; retinal thickness.

Meanwhile, the segmenting neural network can also evaluate probabilities of one or more of the following abnormalities: epiretinal membrane probability; fibrous pigment epithelium detachment probability; geographic atrophy probability; healthy probability (no abnormality or biomarker detected); hard exudates probability; outer retinal atrophy probability; reticular pseudo-drusen probability; drusen probability.

As already mentioned in more generic terms, the above data is then combined with the corresponding non-imaging data such as BCVA, demographic (gender, age) and disease characteristics, as shown at block 702′.

Recurrent neural network (RNN) machine learning algorithms are then applied on the data thus combined at block 702′, to eventually build a predictive model for disease assessment and optimization of dosing regimen, as exemplified at block 703. The above values of measured dimensions of identified anatomical variables and values of probabilities are joined with the non-imaging additional information to form an input data set at time “t”, which can be denoted as D(t). D(t) becomes the input of the above introduced RNNs for outputting a disease assessment score and a dosing regimen recommendation.

Assuming that in the reference clinical trials drug loading weeks are weeks 0, 4 and 8; while non-loading weeks start at week 16 and go on for weeks 20, 24, 28, etc. up to week 96, the mathematical process underlying the generation of the assessment model by application of RNNs for this case can be summarized as in the following sequence of steps.

For a given data sample at a non-loading week “t”, the method according to the present invention takes D(t) as first input data set.

Further to that, a second input dataset, denoted as D(0), is taken, comprising the values of the above identified anatomical variables, probabilities and non-imaging additional information at a baseline loading period “0”.

In a first neural network, D(t) is appended to D(0). The resulting aggregated data are taken as an input to the first neural network and transformed into a first vector space F1(t).

A second neural network computes the difference between D(t) and D(0), denoted as D(t)−D(0), and transforms D(t)−D(0) into a second vector space F2(t).

A third neural network appends F2(t) to F1(t), treats the aggregated data of F1(t) and F2(t) as its input, and transforms this input into a third vector space F3(t).

A fourth neural network transforms F3(t) into a probability score between 0 and 1. Such probability score can be deemed as a disease activity score, as already introduced.

Subsequently, a threshold-based, or benchmark-based, operator determines the data sample as class 1, if the probability score is larger than a given threshold or benchmark. In such case, the system outputs a recommendation to switch from a low frequency dosing regimen to a high frequency dosing regimen, e.g., to switch from a 12-week dosing regimen to a 8-week dosing regimen. Otherwise, the threshold-based operator determines the sample as class 0, that is the system outputs a recommendation to keep the current dosing regimen frequency and to not switch dosing regimen.

Relative to the performance of the RNN-based model as above described, an AuROC measure of 0.835 has been established.

Mutatis mutandis, the abovementioned D(t) can also qualify as an input to a tree-based machine learning method according to the present invention, as previously introduced. In this case, for every feature {xi} in D(t), the machine learning algorithm looks for a function f(xi) predicting the disease activity level and/or the dosing frequency, which can be denoted as y. Mathematically, the function f(xi) needs to approximate to, or predict, a ground truth y, wherein y equals 1 or 0, based on whether the feature {xi} is higher of lower than a threshold value. Graphically, this prediction takes the form of a binary tree: Given every function f(xi), a corresponding weight wi is assigned. The training of the tree-base machine learning algorithms aims at finding the best set of such weights achieving the most accurate prediction. Mathematically, it is required that the true answer y (0 or 1) be as close as possible to the weighted sum of every function f(xi), as signified by the formula y>Σwi f(xi). In an iterative process, the method randomly selects a tree between a current set of trees; turns the selected tree into a child branch of another tree in the given set; evaluates the weighted sum of the prediction of the current tree set-up; and repeats the above steps, until the algorithm performance is optimized. Then, such optimal collection of trees and their respective weights are made part of the ultimate model generated.

A disease activity assessment model generated by tree-based machine learning algorithms according to the present invention identified the 20 most important variables, or features, for outputting an activity disease score, and a correlated dosing regimen recommendation, for example at a target 16th week from beginning of the patient's treatment, as shown in Table 1. In some embodiments, the higher the variables rank, the more weights or the more importantly they contribute to the predictive performance.

In Table 1, the 20 most important variables are ranked by Shapley values (labelled as “Shap” value), measuring the contribution by each feature to the performance of the tree-based model according to the present invention.

The overall tree-based model performance is exemplified in Graph 1, wherein the ROC curve is represented. The area under the ROC curve for the test data set equals 0.88±0.012; whereas the area under the ROC curve for the train data set equals 0.93±0.003.

A tree-based disease activity assessment model according to the present invention can select high-ranked variables to form a reduced, or simplified, set of predictive variables as shown in Table 2, wherein the term “idx” indicates a variable value at the index date when the model is run and the disease activity assessment is carried out, that is at the time of the patient's visit.

The performance of the tree-based model relying on the reduced variable set of Table 2 can be exemplified in Graph 2, wherein the ROC curve is represented. The area under the ROC curve for the test data set equals 0.86±0.001; whereas the area under the ROC curve for the train data set equals 0.9±0.002.

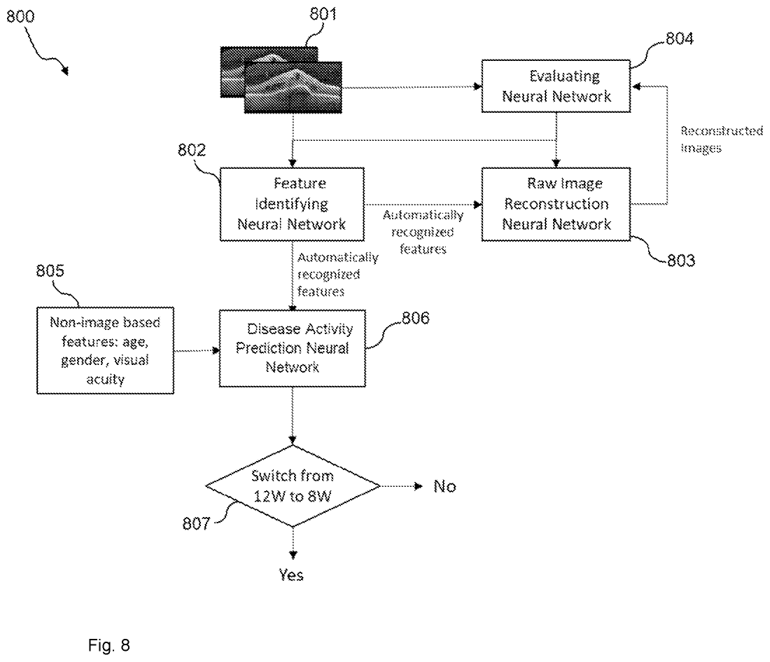

With reference to FIG. 8, it is also shown the workflow for a second approach to the generation of an AI-based clinical decision support software model 700b according to the present invention. This approach follows the path designated by “Deep Learning #2” in FIG. 7.

Such second approach can be defined “feature-agnostic”, in that human users do not pre-determine the exact variables, or features, for classification and/or prediction. Rather, the computational model 700b automatically selects the variables out of the raw data, possibly even automatically deriving the variables itself during the machine learning process.

The model 700b according to such second approach performs therefore end-to-end assessment of disease activity and prediction of disease activity change under a modified dosing regimen, by applying machine learning algorithms to both raw OCT images 701 and non-imaging data.

In short, with reference to FIG. 8, following an analysis of OCT images 801 by a neural network machine 800, a number of features are automatically identified, at block 802; the OCT images are then reconstructed, at block 803; the accuracy of the OCT image reconstruction is evaluated, at block 804, and, based on the neural-network identified features, the system makes a disease activity assessment and/or a prediction of dosing regimen switch, at block 806.

For this purpose, a single neural network architecture 800 comprising a multiplicity of neural networks is employed, namely four neural networks in the specific embodiment exemplified in FIG. 8.

A first neural network, designated Feature Identifying Neural Network takes the raw OCT images 801 as input and generates a number of variables or features, at block 802. A human user can specify the number of features to be generated, such as 100, 200, 240, etc.

A second neural network, designated Raw Image Reconstruction Neural Network, takes the generated features as input, and transforms these features back to the original OCT images 801 as accurately as possible, at block 803.

A third neural network, designated Evaluating Neural Network, evaluates the similarity between the raw OCT images 801 and the reconstructed OCT images, at block 804. If the images are not similar, this neural network asks the previous two neural networks to train; otherwise, the previous two neural networks are considered already properly trained. Once the first and the second neural networks (i.e. Feature Identifying Neural Network and Raw Image Reconstruction Neural Network) are trained, it is assumed that the Feature Identifying Neural Network can automatically recognize features.

A fourth neural network, designated Disease Activity Prediction Neural Network, simultaneously takes the automatically recognized features and other non-image information (such as gender, age and best corrected visual acuity) as input, at block 805, and makes a disease activity assessment. A consequent decision on the suitability of a dosing frequency switch is also output by the Disease Activity Prediction Neural Network, based on the calculated disease activity, at block 807. In fact, the system outputs a recommendation to an ophthalmologist on whether it is appropriate to switch from a low frequency dosing regimen to a high frequency dosing regimen, e.g., to switch from a 12-week dosing regimen to a 8-week dosing regimen; or vice-versa.

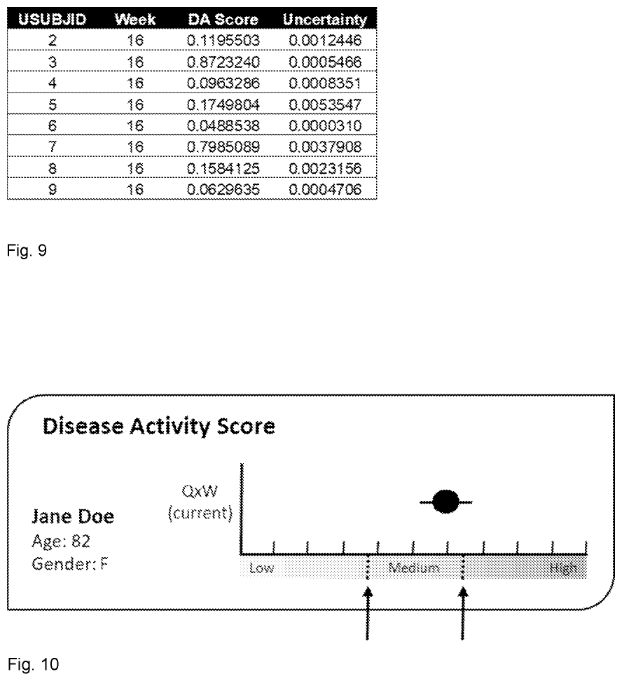

In FIG. 9 it is shown an exemplary set of output data resulting from an application of the computer-implemented method according to the present invention. In particular, an AI-based clinical decision support software implementing such method can output data as shown, yielding disease activity scores, at the target 16th week of treatment, for corresponding 8 anonymized patients identified by numbers “2” to “9”. To each of the activity scores is associated a respective statistical best estimate of how far the calculated disease activity score value might be from its “true value”, that is the uncertainty linked to the calculated score value. It appears evident, from the values output by the model for the disease activity score, how patient 6 has the lowest level of disease activity at the index date, or time of visit; whereas patient 3 has the highest level of disease activity at time of visit.

With reference to the high-level diagram of FIG. 5, a model 500 developed as above described ultimately allows, based on acquisition of a set of input patient data relating to a given patient 10, for instance diagnosed with w-AMD, to output both an assessment of the disease activity and a consequent customized treatment recommendation for the patient 10 to a health care provider or physician 20. The set of input patient data comprises anatomical variables, identified thanks to segmentation 502 of retinal images 501 of the patient 10; and values of clinical, non-image derived input patient data, such as longitudinal pseudonymized electronic medical, or health, records 505 for the same patient 10. As explained, the patient-specific data 501, 505 is elaborated by an AI-based clinical decision support software 500, implementing the method according to the present invention, to calculate disease activity and appropriate dosing frequency as shown in block 503. The output can be for convenience displayed on a health care professional interface 504, as it will be discussed more in detail below.

FIGS. 10 to 13 show examples of portions of graphical user interfaces of a software implementing the method according to the present invention, especially conceived to guide a dosing decision by health care providers, such as ophthalmologists.

It is envisioned that the software graphical user interfaces will provide several levels of information, to guide an ophthalmologist in assessing the disease activity, namely, w-AMD disease activity, during any visit.

A score for disease activity, or DA, will be generated by the model to allow differentiating between levels of DA. Depending on the information generated by the software and read by the ophthalmologist, patients with high DA may be switched to a more frequent dosing; patients with low DA may be maintained under the current dosing regimen or switched to a less frequent dosing (e.g. q8w patients who are well controlled and may be regarded eligible to q12w re-challenge).

To further guide the ophthalmologist in decision making, the relative importance of anatomical and functional features influencing the assessment of DA (e.g. presence and location of retinal fluids) will be displayed.

Finally, OCT images annotated with the relevant anatomical variables as extracted by segmentation will facilitate an immediate validation of the patient's situation.

In particular, FIG. 10 shows a portion of a graphical user interface at a health care professional's or ophthalmologist's front end, for instance on his computer's screen, displaying data output by an AI-based clinical decision support software according to the present invention. The displayed data provides a rendition of current disease activity scores for a patient respectively at two different assessment times e.g. two successive times in the treatment.

The level of disease activity, and of the corresponding score, can be further categorized and labeled as high or medium or low.

In FIG. 10, the patient assessed shows a disease activity which is comprised in an intermediate score region of a rating scale, between a higher end and a lower end thereof. Bounds are shown on the disease activity rating scale which delimit such an intermediate region, or range, of medium disease activity level. Within such range of medium disease activity levels, cases may be more ambiguous for an ophthalmologist to judge. In the case exemplified in FIG. 10, for a patient being treated with an anti-VEGF drug like brolucizumab-dbII in 12-week intervals, the ophthalmologist may still decide to not let the patient switch to a more frequent dosing regimen of 8 week intervals, or Q8W, from a current regimen of 12 week intervals, or Q12W. This may be attributable, for instance, to his/her own general approach e.g. his/her being comfortable with some residual level of disease activity; or because such patient may not be well suited to a more frequent dosing, e.g. because there is a risk of low compliance of the patient to a more intensive treatment. Alternatively, an ophthalmologist, in light of different expert considerations on the treated subject, may want to consider switching to a more frequent dosing regimen of 8 week intervals, e.g. if he/she deems the residual level of disease activity quantified by the model according to the present invention too high a risk.

In FIG. 11 the data displayed provide guidance on suitable drug dosing regimens for a patient examined. In fact, the data show how disease activity levels for the patient's eye can be predicted to change as a result of a change between two different dosing regimens of an anti-VEGF drug to be injected in the patients' eyes. The software predicts that the effects of a more frequent treatment for patient “Jane Doe” may result in a better control of the disease activity. A prediction of disease activity change, namely reduction, is automatically made for a more frequent dosing regimen of 8-week drug intervals between injections of brolucizumab-dbII, compared to a current regimen of 12-week drug injection intervals.

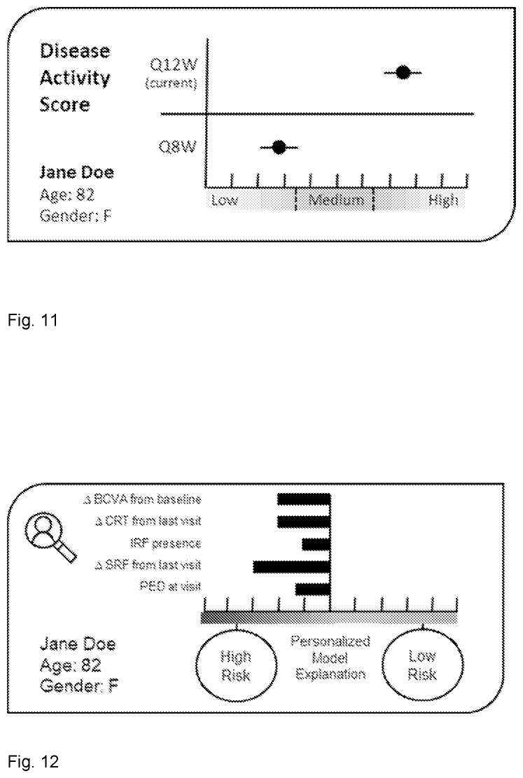

FIG. 12 illustrates how the relative importance of anatomical and functional features influence the assessment of disease activity for the patient of FIG. 11. Main risk factors rendered and quantified are: best corrected visual acuity (BCVA) with respect to a baseline; central retinal thickness (CRT) from last visit; intraretinal fluid (IRF); subretinal fluid (SRF) from last visit; and pigment epithelial detachment (PED) at visit.

In some embodiments, AI-based analysis of fluid volumes from SD-OCT images of patients enrolled in the HAWK and HARRIER clinical trials confirmed that lower levels of IRF, SRF and PED during maintenance were all independently associated with better visual outcomes in terms of BCVA response, showing that all the above-mentioned three fluid compartments are markers of disease activity equally and independently relevant for visual function in w-AMD.

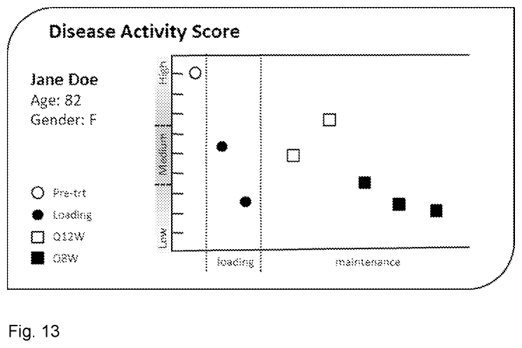

FIG. 13 illustrates a different portion of the graphical user interface of the software implementing the method according to the present invention, wherein the disease activity score for the examined patient is shown over time, from a pre-treatment level to various phases of treatment, across a loading phase carried out with 12-week dosing intervals, and a subsequent maintenance phase with 8-week dosing intervals.

A variety of individualized treatment regimens can be designed and prescribed, based on a disease activity score output by the method according to the present invention. In fact, based on such disease activity score, a health care provider can adjust his/her decisions e.g. to inject a drug immediately, or to assign a next monitoring visits, or to switch to another treatment.

Further examples of application of the method according to the present invention are mentioned in the following.

Under the current label for q12w/q8w treatment with brolucizumab or another anti-VEGF drug, the disease activity score at the current patient's visit, plus information about the time since last injection, can be used by a health care provider to decide whether to switch the patient to a q8w dosing regimen treatment (i.e. an injection every 8 weeks) from the “default” q12w dosing regimen treatment (once every 12 weeks).

Under a “Treat-end-Extend” treatment regimen, the disease activity score at the current patient's visit can be used by a health care provider to decide whether to keep the patient under the current treatment interval, or otherwise to extend or shorten the treatment interval.

Under a “pro-re-nata” or an “as needed” treatment protocol, the disease activity score can be used to discriminate whether to inject an anti-VEGF at a current visit, or to postpone it till next monitoring visit, wherein visits are typically monthly.

Generally, algorithmic assessments of ocular neovascular disease activity in patients, such as in wet-AMD patients, achieved by implementing the method according to the present invention can be used not only for adjusting treatment with anti-VEFG drugs, but also treatment with other drugs designed to maintain vascular stability (for example by suppressing vascular permeability) and/or inhibit angiogenesis, including bispecific antibodies that inhibit both VEGF and angiopoietin-2 (Ang-2).; or even for established laser treatment options.

Moreover, the present invention also relates to an VEGF antagonist for use in the treatment of neovascular age-related macular degeneration (nAMD) in a patient, wherein the use comprises administering to the patient three individual doses of the VEGF antagonist at 4-week intervals, and thereafter administering to the patient an additional dose every 4, 8 or 12 weeks. A 12 week treatment interval can be switched to an 8 week treatment interval if the patient's disease activity is worsening. Otherwise, the 12 week treatment interval can be maintained or extended if the patient's disease is substantially stable or improving. A worsening, an improvement or a stable state of the patient's disease can be advantageously determined based on the computer-implemented method for assessing a level of disease activity, including presence or absence of the disease, above described.

This description and the accompanying drawings that illustrate aspects and embodiments of the present invention should not be taken as limiting the claims defining the protected invention. In other words, while the invention has been illustrated and described in detail in the drawings and foregoing description, such illustration and description are to be considered illustrative or exemplary and not restrictive. Various mechanical, compositional, structural, electrical, and operational changes may be made without departing from the spirit and scope of this description and the claims. In some instances, well-known circuits, structures and techniques have not been shown in detail in order not to obscure the invention. Thus, it will be understood that changes and modifications may be made by those of ordinary skill within the scope and spirit of the following claims. In particular, the present invention covers further embodiments with any combination of features from different embodiments described above and below.

The disclosure also covers all further features shown in the Figs. individually although they may not have been described in the afore or following description. Also, single alternatives of the embodiments described in the figures and the description and single alternatives of features thereof can be disclaimed from the subject matter of the invention or from disclosed subject matter. The disclosure comprises subject matter consisting of the features defined in the claims or the exemplary embodiments as well as subject matter comprising said features.

Furthermore, in the claims the word “comprising” does not exclude other elements or steps, and the indefinite article “a” or “an” does not exclude a plurality. A single unit or step may fulfil the functions of several features recited in the claims. The mere fact that certain measures are recited in mutually different dependent claims does not indicate that a combination of these measures cannot be used to advantage. The terms “essentially”, “about”, “approximately” and the like in connection with an attribute or a value particularly also define exactly the attribute or exactly the value, respectively. The term “about” in the context of a given numerate value or range refers to a value or range that is, e.g., within 20%, within 10%, within 5%, or within 2% of the given value or range. Components described as coupled or connected may be electrically or mechanically directly coupled, or they may be indirectly coupled via one or more intermediate components. Any reference signs in the claims should not be construed as limiting the scope.

1. A computer-implemented method for assessing a level of activity, including presence or an absence, of a disease in at least one eye of a patient, wherein the disease is a neovascular ocular disease;
the method comprising the steps of:
at one or more computing devices, comprising one or more processors, a memory and one or more input and/or output elements:
receiving, via the one or more input elements, a set of input patient data corresponding to the patient, the set of input patient data comprising at least one or more retinal images of the patient;
applying a first algorithm for imaging data analysis to the one or more retinal images to identify, based on the one or more retinal images, values of one or more anatomical variables of the patient's eye;
applying a second algorithm
to the values of the one or more anatomical variables identified, and
to distinct clinical, non-image derived input patient data comprised in the set of input patient data;
making an assessment of the level of activity of the disease in the at least one eye of the patient, and/or of the progression or regression of the disease with respect to a level of activity formerly determined, based on the application of the second algorithm;
wherein the assessment of disease activity corresponds to a dosing regimen of a drug for treatment of the patient's eye disease; and
based on the assessment, generating and outputting, via the one or more output elements, a disease activity score corresponding to the level of activity of the disease

2. The method of claim 1, comprising a step of determining a prediction of disease activity level as a result of a change from a current dosing regimen of the drug for treatment of the patient's eye disease to a different dosing regimen thereof

3. The method of claim 2, wherein the disease activity score is linked to the probability, or appropriateness, of switching between different dosing regimens.

4. The method of claim 2, wherein determining the prediction of disease activity level is based on predicting a physiological change in one of the one or more identified anatomical variables over a period of time.

5. The method of claim 2, wherein the dosing regimen comprises a drug administration frequency for treating the patient with a dose of the drug, the method further comprising the step of:
generating, using a third algorithm, a drug administration frequency recommendation, based on the values of the one or more anatomical variables identified and/or based on the disease activity score.

6. The method of claim 5, wherein generating the drug administration frequency recommendation comprises the steps of:
generating, using the third algorithm, one or more probabilistic simulations of treatment outcomes for different drug dosing regimens; and
generating the drug administration frequency recommendation based on the one or more probabilistic simulations of treatment outcomes.

7. The method of claim 6, further comprising the steps of:
generating, using the third algorithm, a prediction of time-dependent visual acuity gain based on the generated drug administration frequency recommendation.

8. The method of claim 5, wherein the drug administration frequency recommendation includes a parameter selected from the group consisting of:
a drug dosing frequency interval;
a next drug dosing date;
a next drug dose amount;
a next retinal imaging date;
a next visit date for monitoring of the patient by a health care provider; and/or
a combination thereof.

9. The method of claim 8, wherein the set of input patient data further comprises data selected from a group consisting of:
patient longitudinal data on visual acuity; patient longitudinal data on physiological characteristics; previous disease activity scores; previous drug administration frequency recommendations; and/or a combination thereof.

10. The method of claim 1, wherein the one or more anatomical variables are selected from the group consisting of: central retinal thickness and/or volume; sub-retinal fluid volume; inter-retinal fluid volume; pigment epithelial detachment, also indicated as PED; drusenoid, fibrovascular, or serous PED; hyperreflective foci; ellipsoid zone defect; external limiting membrane band defect; retinal pigment epithelial atrophy; central subfield foveal thickness (CSFT); ganglion cell layer and inner plexiform layer; inner nuclear layer and outer plexiform layer volume; cysts volume; outer nuclear layer volume; pigment epithelium detachment volume; photoreceptors and retinal pigment volume; retinal nerve fiber layer volume; and/or a combination thereof.

11. The method of claim 1, wherein at least one of the one or more retinal images is an optical coherence tomography (OCT) image.

12. The method of claim 11, wherein the OCT image is generated by a spectral domain optical coherence tomography (SD-OCT) imaging device.

13. The method of claim 1, wherein the second algorithm is a disease activity assessment model generated by one or more machine learning algorithms comprising a multiplicity of input variables corresponding to the one or more identified anatomical variables and to the distinct clinical, non-image derived input patient data;
the one or more machine learning algorithms being trained on a historical set of patient data from a plurality of historical patients diagnosed with the disease;
the historical set of patient data including input values for the one or more identified anatomical variables, derived from retinal images of the historical patients; and/or including values correlated to clinical, non-image derived input patient data, comprising historical patients' demographics and/or medical history and/or concomitant medication and/or comorbidities and/or adverse events and/or serious adverse events.

14. The method of claim 13, wherein the historical set of patient data comprises input values extracted from at least one of clinical trial data and anonymized real-world patient data from commercially-available databases.

15. The method of claim 14, further comprising the steps of:
updating the second algorithm based on a further historical set of patient data from a further plurality of historical patients diagnosed with the disease.

16. The method of claim 15, wherein updating the second algorithm comprises the step of:
re-training the one or more machine learning algorithms by complementing the historical set of patient data with the further historical set of patient data; and
generating, with the one or more re-trained machine learning algorithms, an updated disease activity assessment model.

17. The method of claim 15 or 16, wherein the disease assessment model is updated in real time, the further historical set of patient data comprising real-world patient data employed for assessing the level of activity of the disease and/or the progression or regression of the disease in corresponding real-world patients.

18. The method of claim 17, wherein the real-world patient data comprises updated anatomical variable data, such as change in anatomical variable measurements, over a period of time.

19. The method of claim 1, wherein the drug for treating the patient's eye disease inhibits vascular endothelial growth factor (VEGF), wherein the drug is an anti-VEGF drug.

20. The method of claim 14, wherein the clinical trial data comprise data associated with one or more anti-VEGF drugs and effect thereof on at least one of the one or more identified anatomical variables.

21. The method of claim 1, wherein the patient's eye disease is one of:
wet age-related macular degeneration, also designatable as w-AMD;
diabetic retinopathy, also designatable as DR; and/or
diabetic macular edema, also designatable as DME; and/or
myopic choroidal neovascularization, also designatable as mCNV; and/or
macular edema following retinal vein occlusion, also designatable as RVO.

22. The method of claim 1, wherein the first algorithm and/or the second algorithm and/or the third algorithm are machine learning generated models comprising a gradient boosted decision trees algorithm, such as a LightGBM or an XGBoost algorithm; and/or a Recurrent neural network algorithm.

23. A system comprising a computing device including:
one or more processors;
one or more input and/or output elements;
memory; and
one or more programs stored in the memory, the one or more programs including instructions for:
receiving, via the one or more input elements, a set of input patient data corresponding to a patient affected by a neovascular ocular disease, the set of input patient data comprising at least one or more retinal images of the patient;
applying a first algorithm for imaging data analysis to the one or more retinal images to identify, based on the one or more retinal images, values of one or more anatomical variables of the patient's eye;
applying a second algorithm to the values of the one or more anatomical variables identified and to distinct clinical, non-image derived input patient data of the set of input patient data;
making an assessment of the level of activity of the disease in the at least one eye of the patient, and/or of the progression or regression of the disease with respect to a level of activity formerly determined for the patient, based on the application of the second algorithm;
wherein the assessment of disease activity corresponds to a dosing regimen of a drug for treatment of the patient's eye disease; and
based on the assessment, generating and outputting, via the one or more output elements, a disease activity score corresponding to the level of activity of the disease.

24. (canceled)

25. A non-transitory computer-readable storage medium storing one or more programs configured to be executed by one or more processors of a computing system, the one or more programs including instructions for:
receiving, via the one or more input elements, a set of input patient data corresponding to a patient affected by a neovascular ocular disease, the set of input patient data comprising at least one or more retinal images of the patient;
applying a first algorithm for imaging data analysis to the one or more retinal images to identify, based on the one or more retinal images, values of one or more anatomical variables of the patient's eye;
applying a second algorithm to the values of the one or more anatomical variables identified and to distinct clinical, non-image derived input patient data of the set of input patient data;
making an assessment of the level of activity of the disease in the at least one eye of the patient, and/or of the progression or regression of the disease with respect to a level of activity formerly determined for the patient, based on the application of the second algorithm;
wherein the assessment of disease activity corresponds to a dosing regimen of a drug for treatment of the patient's eye disease; and
based on the assessment, generating and outputting, via the one or more output elements, a disease activity score corresponding to the level of activity of the disease.

26. (canceled)

27. A computer-implemented method for assessing a level of activity, including presence or an absence, of a disease in at least one eye of a patient, wherein the disease is a neovascular ocular disease;
the method comprising the steps of:
at one or more computing devices, comprising one or more processors, a memory and one or more input and/or output elements:
receiving, via the one or more input elements, a set of input patient data corresponding to the patient, the set of input patient data comprising at least one or more retinal images of the patient;
applying an algorithm simultaneously both to the one or more retinal images and to non-image data components to make an assessment of the level of activity of the disease in the at least one eye of the patient, and/or of the progression or regression of the disease with respect to a level of activity formerly determined for the patient;
wherein the assessment of disease activity corresponds to a dosing regimen of a drug for treatment of the patient's eye disease; and
based on the assessment, generating and outputting, via the one or more output elements, a disease activity score corresponding to the level of activity of the disease.

28. An VEGF antagonist for use in the treatment of neovascular age-related macular degeneration (nAMD, or w-AMD) in a patient, the use comprising administering to the patient three individual doses of the VEGF antagonist at 4-week intervals, and thereafter administering to the patient an additional dose every 12 weeks; wherein a 12 week treatment interval is switched to an 8 week treatment interval if the patient's disease activity is worsening; or a 12 week treatment interval is maintained if the patient's disease is substantially stable or improving; wherein a worsening, an improvement or a stable state of the patient's disease is determined based on the computer-implemented method for assessing a level of disease activity, including presence or absence of the disease, according to claim 27.
